(19) United States
(12) Patent Application Publication
(10) Pub. No.: US 2022/0330533 A1
(43) Pub. Date: Oct. 20, 2022

(54) SYSTEMS AND METHODS FOR BATCH CULTIVATION OF NON-TRANSGENIC HETEROGAMETES

(71) Applicant: REGENTS OF THE UNIVERSITY OF MINNESOTA, Minneapolis, MN (US)

(72) Inventors: Michael Joseph SMANSKI, Falcon Heights, MN (US); Siba DAS, Falcon Heights, MN (US); Maciej MASELKO, Sydney (AU)

(21) Appl. No.: 17/763,849

(22) PCT Filed: Oct. 1, 2020
(86) PCT No.: PCT/US2020/053749

§ 371 Date: Mar. 25, 2022

Related U.S. Application Data
(60) Provisional application No. 62/909,536, filed on Oct. 2, 2019.

(51) Int. Cl. A01K 67/033; C12N 15/85

(52) U.S. Cl. CPC A01K 67/0339; C12N 15/8509; C12N 2800/105; C12N 2830/006; C12N 2800/90; A01K 2217/05; A01K 2217/203; A01K 2227/706

(57) ABSTRACT

A system for batch production of the heterogametic sex of a biological species generally includes a first strain of a biological species genetically engineered to include a conditional Y-linked (or Z-linked) genetic lethal circuit and a second strain of the biological species genetically engineered to include a conditional X-linked (or W-linked) genetic lethal circuit.

### CROSS-REFERENCE TO RELATED APPLICATION

This application claims the benefit of U.S. Provisional Patent Application No. 62/909,536, filed Oct. 2, 2019, which is incorporated herein by reference in its entirety.

### GOVERNMENT FUNDING

This invention was made with government support under Grant No. HR0011836772 awarded by the Department of Defense/Defense Advanced Research Projects Agency (DARPA). The government has certain rights in the invention.

### SEQUENCE LISTING

This application contains a Sequence Listing electronically submitted to the United States Patent and Trademark Office via EFS-Web as an ASCII text file entitled “Seq_Listing-0110-000629_ST25.txt” having a size of 30 kilobytes and created on Sep. 29, 2020. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a system that includes a first strain of a biological species genetically engineered to include a conditional Y-linked genetic lethal circuit and a second strain of the biological species genetically engineered to include a conditional X-linked genetic lethal circuit. The system may be used to selectively produce non-transgenic males.

In some embodiments, the X-linked genetic lethal circuit is the same as the Y-linked genetic lethal circuit.

In some embodiments, the X-linked genetic lethal circuit is different than the Y-linked genetic lethal circuit.

In some embodiments, the biological species is a pest species.

In some embodiments, the biological species is a species in which one sex has greater commercial value than the other sex.

In another aspect, this disclosure describes a method of selecting non-transgenic males of a biological species. Generally, the method includes providing a first strain of the biological species genetically engineered to have a conditional Y-linked genetic lethal circuit; providing a second strain of the biological species genetically engineered to have a conditional X-linked genetic lethal circuit; performing a first cross mating males of the first strain and females of the first strain under conditions effective to express the conditional Y-linked genetic lethal circuit, thereby producing non-transgenic female progeny, then mating the non-transgenic progeny of the first cross with males of the second strain under conditions effective to express the conditional X-linked genetic lethal circuit, thereby producing non-transgenic males.

In some embodiments, the X-linked genetic lethal circuit is the same as the Y-linked genetic lethal circuit.

In some embodiments, the X-linked genetic lethal circuit is different than the Y-linked genetic lethal circuit.

In some embodiments, the biological species is a pest species.

In some embodiments, the biological species is a species in which one sex has greater commercial value than the other sex.

In some embodiments, the method further includes subjecting the non-transgenic males to a treatment effective to sterilize the males. In some of these embodiments, the males are sterilized by subjecting the males to X-ray irradiation.

In another aspect, this disclosure describes a system that includes a first strain of a biological species genetically engineered to have a conditional W-linked genetic lethal circuit and a second strain of the biological species genetically engineered to have a conditional Z-linked genetic lethal circuit. The system may be used to selectively produce non-transgenic females.

In some embodiments, the Z-linked genetic lethal circuit is the same as the W-linked genetic lethal circuit.

In some embodiments, the Z-linked genetic lethal circuit is different than the W-linked genetic lethal circuit.

In some embodiments, the biological species is a pest species.

In some embodiments, the biological species is a species in which one sex has greater commercial value than the other sex.

In another aspect, this disclosure describes a method of selecting non-transgenic females of a biological species. Generally, the method includes providing a first strain of the biological species genetically engineered to have a conditional W-linked genetic lethal circuit, providing a second strain of the biological species genetically engineered to have a conditional Z-linked genetic lethal circuit, performing a first cross mating males of the first strain and females of the first strain under conditions effective to express the conditional W-linked genetic lethal circuit, thereby producing non-transgenic male progeny, and mating the non-transgenic progeny of the first cross with females of the second strain under conditions effective to express the conditional Z-linked genetic lethal circuit, thereby producing non-transgenic females.

In some embodiments, the Z-linked genetic lethal circuit is the same as the W-linked genetic lethal circuit.

In some embodiments, the Z-linked genetic lethal circuit is different than the W-linked genetic lethal circuit.

In some embodiments, the biological species is a pest species.

In some embodiments, the biological species is a species in which one sex has greater commercial value than the other sex.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Overview of STSS. Minimal requirements for each strain to be used in STSS, including true-breeding population with conditional Y-linked lethality or conditional X-linked lethality. (A) Schematic illustration of Y-linked lethality. (B) Schematic illustration of X-linked lethality. (C) Mating scheme in absence of lethal gene repressor. Combining non-transgenic females produced from the Y^{L} strain with adult flies from the X^{L} strain results in death of all offspring except for non-transgenic males. This includes offspring from mating events of X^{L} males and females. Tet, tetracycline.

FIG. 2. Application of STSS in sex selection of chicken. (A) In chicken, females determine the sex of the offspring. A conditional Z-linked lethal strain would allow maintenance of the strain in presence of the permissible medium. (B) Mating scheme in absence of lethal gene repressor. Mating between wildtype males and Z-linked lethal strain females results in death of all offspring except for non-transgenic males.

FIG. 3. Sex-chromosome linked tet-repressible lethal circuits are effective. (A) Construct-level schematic of tet-repressible lethal circuit used in this study. (B) Proportion of male and female offspring generated from self-mating of DmX^{L-tTA} or with non-transgenic (w1118) flies in presence or absence of tetracycline. Genotypes of parental files are indicated below x-axis. Numbers above bars indicate total number of progeny produced from six biological replicates. (C) Proportion of male and females generated from mating DmY^{L-tTA} with non-transgenic (w1118) flies in presence or absence of tetracycline. Numbers above bars show total number of progeny produced from three biological replicates. * indicates statistically significant difference from expected 50:50 male:female sex ratio (chi-squared test, p<0.05). ** indicates a statistically significant difference between the +tet and -tet groups (chi-squared test, p<0.001).

FIG. 4. Batch production of adult males via STSS. (A) Average number of adult males obtained from mating between different proportions of non-transgenic female flies obtained from DmY^{L-tTA} in the absence of tetracycline (‘XX*’) when combined with adult DmX^{L-tTA} flies in absence of tetracycline. Data represent mean numbers from two or three biological replicates with error bars showing standard deviation. Average numbers of females produced are indicated numerically above bars. Numbers below x-axis indicate ratio of genotypes in parental generation. Total numbers from all replicates are (from left to right): N=822, N=1151, N=1292, N=1301, N=1086, N=1078. (B) Bright-field (left) and fluorescent (right) images of parental (i) 10 DmY^{L-tTA} females produced in the absence of tetracycline (‘XX*’), parental (ii) 10 DmX^{L-tTA} males, and (iii) approximately 400 offspring from the batch production of non-transgenic males. Files in (i) and (ii) are only included for visual comparison to the STSS males; they were not present in the final batch of STSS flies. (C) PCR amplification of transgene cassette from genomic DNA isolated from 10 DmX^{L-tTA} flies (+), 1180 batch-produced STSS males (male symbol), or STSS gDNA spiked with DNA from DmX^{L-tTA} flies at five-fold dilutions from 1:5 (right) to 1:3125 (left). L denotes 1 kb plus DNA ladder (ThermoFisher Scientific, Inc., Waltham, Mass.).

FIG. 5. An exemplary approach of STSS. (A) Reproductive behavior of X-linked Female Lethal construct used in female-lethal (FL-) STSS. (B) Mating scheme for producing non-transgenic males via FL-STSS. Combining non-transgenic females produced from the YL strain with adult male flies produced from the X^{FL} strain results in death of all offspring except for non-transgenic males. (C) Genetic design of FL construct.

FIG. 6. An exemplary approach of STSS. (A) Chromosomal location of FL constructs in two copy ‘FL12a-c’ flies. FL1 and FL2 have only one copy of the X-linked FL construct on their X-chromosome. (B) Proportion of male and female offspring generated from self-mating or outcrosses to wild-type (w1118) for DmX^{FL1}, DmX^{FL2}, and three independently generated DmX^{FL12} genotypes. Parental genotypes are indicated below the x-axis. Results are shown in the presence or absence of tetracycline. Numbers above bars indicate total number of progeny produced from at least three biological replicates. (C) Average number of adult males obtained from mating between different proportions of non-transgenic female flies obtained from DmY^{L-tTA} in absence of tetracycline (′XX*′) when combined with adult male DmX^{FL12c} flies in absence of tetracycline. Data represent mean numbers from 2 biological replicates with error bars showing standard deviation. Average numbers of females produced are indicated numerically above bars. Total numbers from all replicates are (from left to right): N=460, N=475, N=692, N=617. Numbers below x-axis indicate number of parental flies of each genotype. * indicates statistically significant difference from expected 50:50 male:female sex ratio (chi-squared test, p<0.05). ** indicates a statistically significant difference between the +tet and -tet groups (chi-squared test, p<0.001).

FIG. 7. Characterization of unexpected flies. Obtained female (♀*) from the final mating and male (♂*) from DmY^{L-tTA} mating in absence of tetracycline were assayed for the presence of X and Y-chromosome by amplifying an X-chromosome specific gene, upd1 and Y-chromosome specific gene, kl-5. L denotes 1 kb plus DNA ladder (Thermo Fisher Scientific, Waltham, Mass.).

FIG. 8. An example of an inducible lethal system. A temperature sensitive promoter (pHsp70Bb) induces overexpression of a gene (hedgehog; Hh) that results in lethality in response to elevated temperature.

### DETAILED DESCRIPTION OF ILLUSTRATIVE EMBODIMENTS

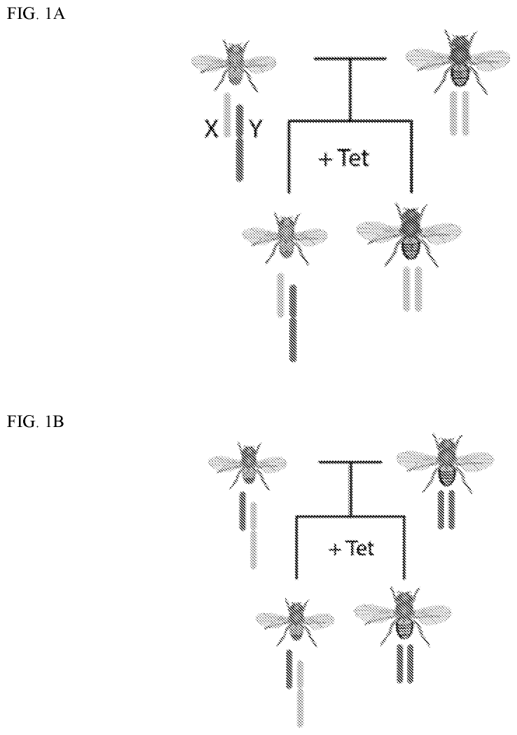

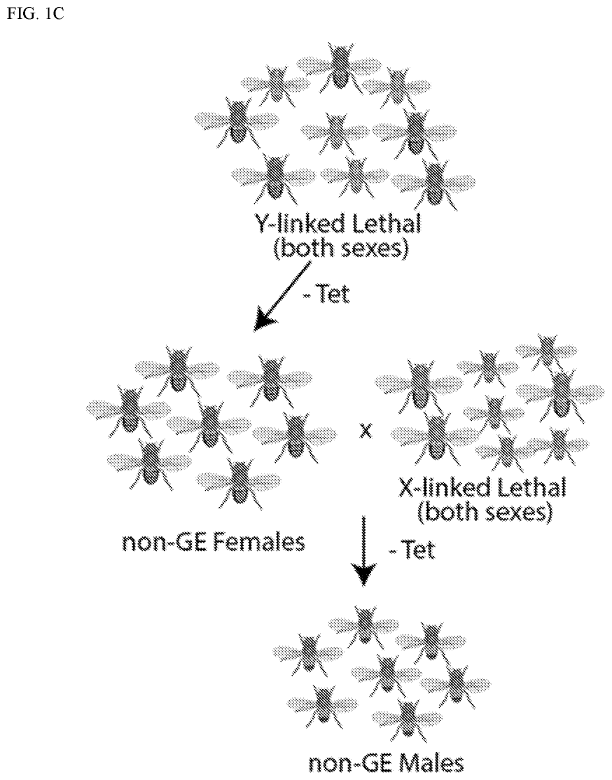

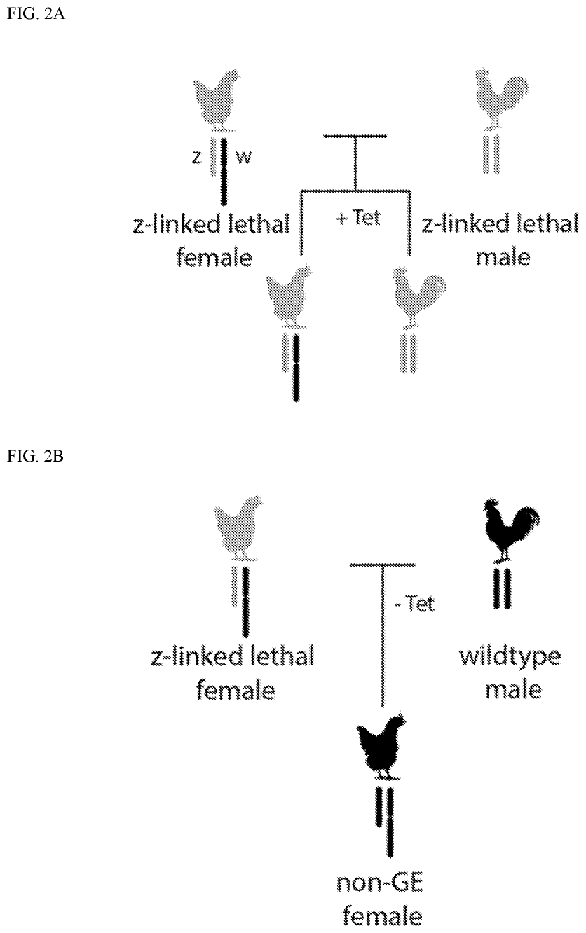

This disclosure describes and demonstrates systems and methods for batch production of the heterogametic sex of a species that are suitable for, for example, sterile pest control. In many species, the heterogametic sex is male (XY, FIG. 1). In other species, the heterogametic sex is female (WZ, FIG. 2). The systems and methods described herein may be applied to generate the heterogametic sex (XY or WZ) in any organism with chromosomal sex determination. At least 99% sex-selection was observed with batch cultures as large as 6800 individuals. Transgenes were not detected in the surviving progeny.

Insect pests impose a major burden to food production and human health worldwide. The most successful population control method in use today is the sterile insect technique (SIT). SIT relies on mass rearing of pest insects followed by a sterilization treatment (e.g., X-ray irradiation). Sterilized insects are released into the wild where sterile males compete with wild males to seek out and mate with wild females. The female of many pest insects will typically only mate once in her lifetime. Mating with a sterile male therefore prevents successful reproduction. Sufficiently large releases of sterile insects can be used to eliminate wild populations or prevent their introduction in an area at threat of their introduction. Also, SIT is considered safe to humans and the environment, as there are fewer off-target effects compared to the application of chemical pesticides.

Existing SIT programs are used to control several major agricultural pests including the New World Screwworm (Cochliomyia hominivorax), Mediterranean Fruit Fly (Ceratitis capitata), and Queensland Fruit Fly (Bactrocera tryoni). All together, these programs produce and release billions of sterile insects on a weekly basis. SIT for many insects, including C. hominivorax and B. tryoni, currently involves releasing both sterilized males and females. However, the effectiveness of SIT can be substantially increased if only males are released since they will then seek out wild females instead of mating with co-released sterile females. In some insect pests, such as the Yellow Fever Mosquito (Aedes aegypti), SIT programs only release sterile males since sterile females can vector disease.

A variety of sex-sorting techniques have been developed. Mechanical separation of Aedes aegypti pupae based on size differences can be effective and flow cytometric separation of transgene-expressing female Anopholes gambiae has been demonstrated. These approaches can be labor intensive and/or require sophisticated equipment, however. Combining irradiation with temperature-sensitive lethal (tsl) mutant strains of C. capitata is presently in use in SIT programs. Repressible transgenic female-elimination constructs act as genetic biocontrol systems on their own and have been developed for Ae. aegypti, C. hominivorax, Sheep Blow Fly (Lucilia cuprina), Diamondback moth (Plutella xylostella), Pink Bollworm (Pectinophora gossypiella), and Silkworm (Bombyx mori). Despite their effectiveness, specificity, reduction of insecticide use, and safety, public resistance and regulatory hurdles have limited the broad use of released transgenic insects for pest control.

The sex selection methods described herein may have applications beyond controlling pest species. For example, selection of female-only progeny is desirable in egg-layer poultry production since there is little economical need for the male chicks in the egg-layer industry. Current practices of female-only chicken selection use physical characteristics in day-old chicks. In some cases, the separated male chicks are culled and discarded, often subjected to industrial grade mechanical grinders. This practice is banned in several countries as being inhumane. It is also laborious and error-prone. Other methods for in-egg sex selection require individual screening of eggs with sophisticated and time-consuming instrumentation. The methods described herein provide a humane, labor-free, and accurate strategy for sex selection in egg-laying poultry.

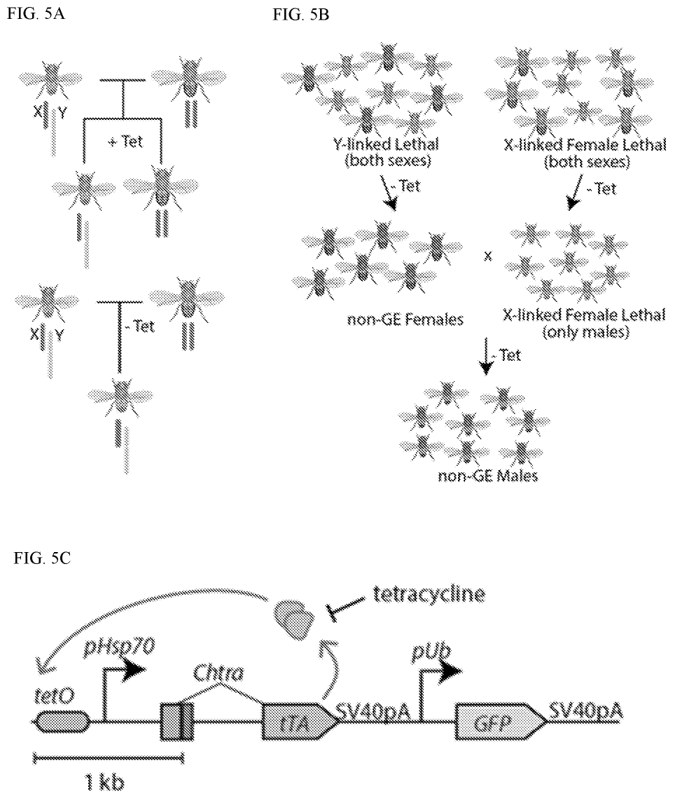

This disclosure describes a genetic approach to produce non-transgenic males in Drosophila melanogaster, referred to herein as Subtractive Transgene Sex Sorting (STSS). STSS relies on two transgenic strains, each of which has bi-sex lethal genetic circuit that can be induced or repressed. One of the strains has the lethal circuit on the Y-chromosome (Y^{L} strain, FIG. 1A) and the other has the lethal circuit on the X-chromosome (X^{L} strain, FIG. 1B). Non-transgenic males are produced (FIG. 1C) by first switching the Y^{L} strain to media that activates the lethal circuit, resulting in non-transgenic females. These non-transgenic females are combined with the X^{L} strain in media that activates the lethal circuit. Mating between the X^{L} males and non-transgenic females results in non-transgenic males. All other offspring die. An alternative approach is to create a strain containing X-linked female lethal circuit (FL-STSS, FIG. 5A), which when active is selectively lethal to females containing the circuit. Males produced from this strain in the selection media when crossed with females obtained from Y^{L} strain would result in non-transgenic males (FIG. 5B). This technique is transferable to any organism that relies on genetic, as opposed to environmental, sex determination (Smanski MJ & Zarkower, D. 2019. EMBO Reports 20:e48577).

### Design and Construction of a Repressible Lethal and Female-Specific Lethal Transgenic Construct

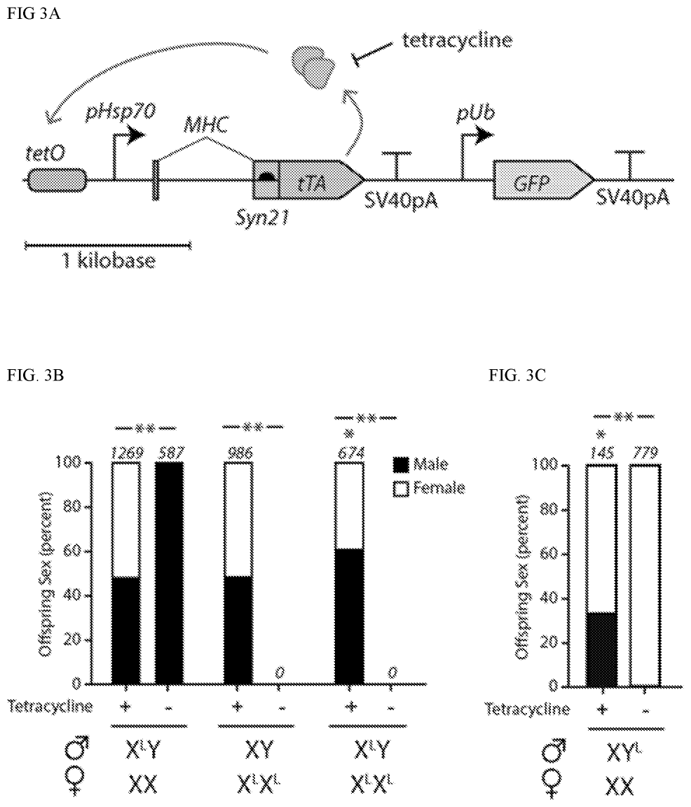

A repressible lethal genetic construct can be designed with a conditionally-expressed promoter driving a toxic gene product. Two exemplary constructs are illustrated in FIG. 3A and FIG. 5C. A tetracycline-repressible hsp70 minimal promoter (pHsp70) was selected due to its well-characterized behavior in model and applied insect species. To drive lethality, the tet-transactivator (tTA) was expressed, the VP64 transactivation domain of which is toxic to cells when expressed strongly. Plasmids were constructed with a positive feedback loop where the hsp70 minimal promoter drives basal expression of the tet-transactivator (tTA) similar to what has been previously described (Shockett et al., 1995, Proc. Natl. Acad. Sci. USA, 92:6522-6526). In the absence of tetracycline, tTA binds to operators upstream of the hsp70 minimal promoter and establishes a positive feedback loop that generates lethal amounts of tTA. 5′ UTR translational enhancer features were incorporated to further boost tTA expression. Tuning gene expression in lethal transgenic constructs is a balance between (i) incurring fitness effects from leaky expression in the repressed ‘off’-state and (ii) incomplete penetrance due to weak expression in the de-repressed ‘on’-state. To ensure complete penetrance of the lethal phenotype in the derepressed state, the construct was designed to favor strong expression.

PhiC31-mediated transgenesis was used to integrate a single copy of the tTA circuit into AttP landing sites on the X and Y chromosomes of two separate strains (referred to as DmX^{L-tTA} and DmY^{L-tTA} from here on). Both of these strains were maintained in the presence of 200 μg/ml tetracycline. The genotype of transgenic flies was confirmed by PCR amplification and Sanger sequencing of engineered loci. The DmX^{L-tTA} were mated with a X-chromosome balancer strain and then selfed to screen for females homozygous for the modified X-chromosome, which was confirmed by PCR. From this point on, DmX^{L-tTA} and DmY^{L-tTA} were maintained as true-breeding lines in the presence of tetracycline.

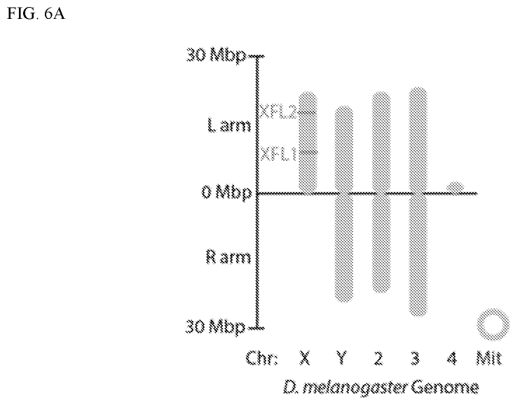

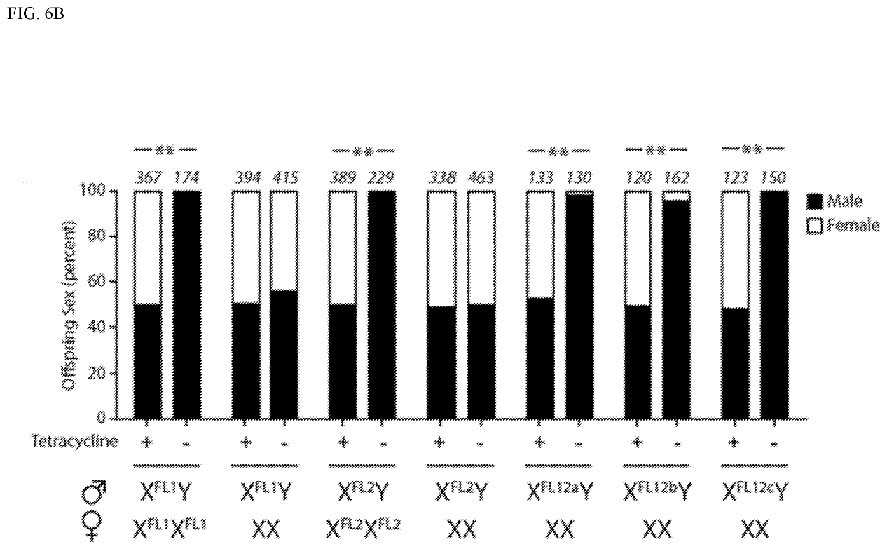

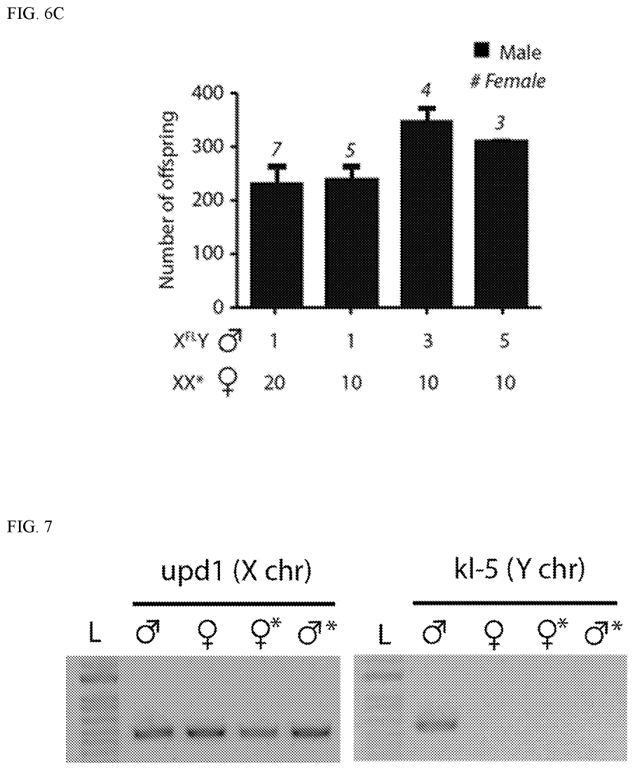

Similar methods were used to create X^{FL} strain with the exception of there being insertions into two separate locations in the X-chromosome (FIG. 6A), referred to as DmX^{FL1} and DmX^{FL2}. Both DmX^{FL1} and DmX^{FL2} were maintained as homozygous true breeding lines in the presence of 10 μg/ml tetracycline. In order to create a line containing inserts in both the locations of the X-chromosome, DmX^{FL1} and DmX^{FL2} were crossed with each other and several recombinants were isolated and identified by screening with PCR. They were balanced and selfed to create homozygous true breeding line and maintained in presence of 10 μg/ml tetracycline.

### Performance of Repressible Lethal and Female Lethal Genetic Constructs

To test the efficiency of toxic gene expression, virgin females and males were mated on media lacking tetracycline. In each of three replicate crosses, no DmX^{L-tTA} adults survived to adulthood (FIG. 3B). This suggests that the repressible lethal transgenic construct is sufficiently strong to cause lethality in two copies (females) or one copy (males). In an analogous experiment with DmY^{L-tTA} flies ten replicate crosses produced 777 females and only two males (99.7% females, FIG. 3C). These males did not reproduce when subsequently mated with non-transgenic females and lacked a Y chromosome (XO, FIG. 7), likely a result of nondisjunction. Thus, both the DmX^{L-tTA} and DmY^{L-tTA} produced a sufficiently lethal phenotype in the absence of tetracycline to remove the transgene from the accessible gene pool (FIG. 3B, 3C). Both DmX^{FL1} and DmX^{FL2} strains were equally efficient at producing 100% males in absence of tetracycline only as true breeding line (FIG. 6B). To use a heterozygote DmX^{FL} for biocontrol, the two DmX^{FL1} and DmX^{FL2} lines were combined. Three independent lines containing transgene in the two locations (DmX^{FL12a}, DmX^{FL12b}, DmX^{FL12c}) produced almost 100% males in absence of tetracycline as a heterozygote (FIG. 6B). The females produced in absence of tetracycline were very sick and never produced any progeny.

Sub-Stoichiometric Ratio of Mixed-Sex DmXL^{tTA} to Female DmYL^{tTA} Sufficient for Non-Transgenic Male Production

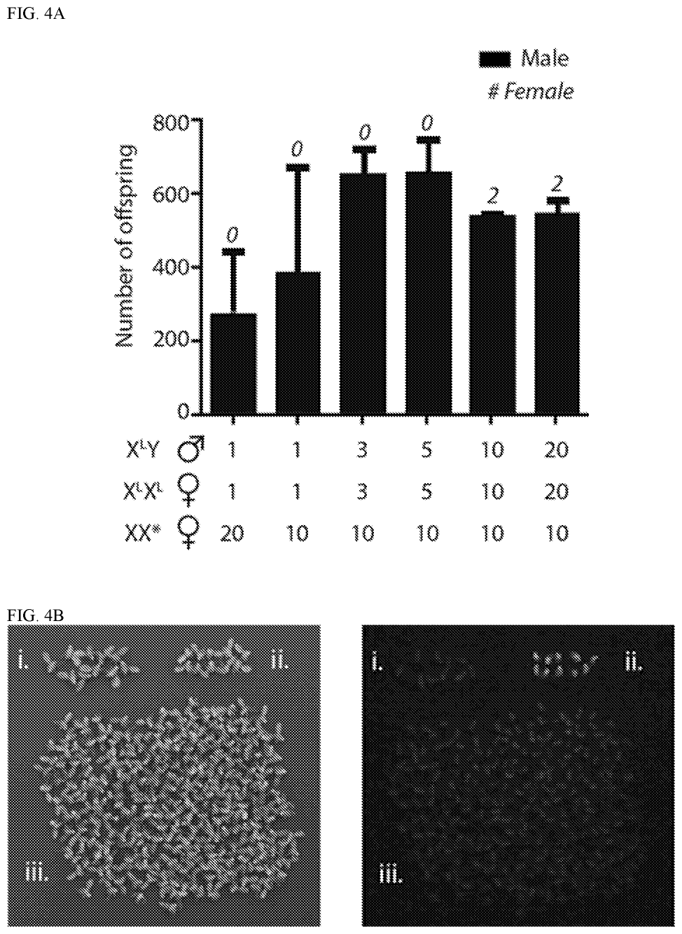

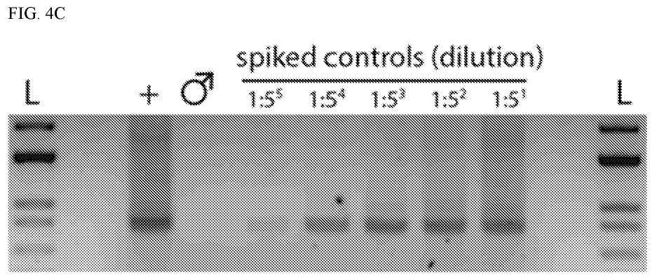

Non-transgenic males can be generated by crossing non-transgenic females produced by the DmY^{L-tTA} strain and males from a mixed-sex true-breeding population of DmX^{L-tTA} flies (FIG. 1C). The number of non-transgenic males produced is directly related to the number of non-transgenic mothers, but is unaffected by decreasing numbers of DmX^{L-tTA} fathers. This would be important for economically scaling-up the production of non-transgenic males for SIT programs. Experimental crosses were performed between non-transgenic females and DmX^{L-tTA} mixed-sex populations to determine the minimum sufficient ratio of parental genotypes. A monotonically increasing number of total offspring were produced as the ratio of DmX^{L-tTA} males to DmY^{L-tTA} females increased from 1:20 to 3:10 (FIG. 4A). The offspring number appeared to plateau or even decline after further increasing the number of DmX^{L-tTA} males. This suggests that a ˜1:3 ratio is sufficient to ensure that the number of DmX^{L-tTA} males are not limiting the total number of offspring produced.

At or below the optimal ratios of DmX^{L-tTA} males to DmY^{L-tTA} females, 100% male offspring (N_{combined}=5388 male offspring, 0 female offspring) were observed (FIG. 4A). A total of four female offspring across all replicates when the ratio of DmX^{L-tTA} males to DmY^{L-tTA} females was 10:10 or 20:10 (FIG. 4A; N_{combined}=2142 male offspring, 4 female offspring). It is unclear how these females were able to survive, but they lacked a GFP phenotype, did not appear to be transgenic, and did not carry a Y chromosome (FIG. 7), indicating that they were not XXY females.

An alternative approach of creating STSS (FIG. 5A, 5B), where two copies of the female lethal construct illustrated in FIG. 5C are inserted into two different locations of X-chromosome (FIG. 6A) results in similar number of males and similar percentage of males from different ratios of GE males and non-GE females (FIG. 6B, 6C). Females observed in this method were very sick and failed to produce viable progeny when crossed with healthy wildtype males.

### Large-Scale Cultivation of Non-Transgenic Males Suitable for Egg Release

Next, the effectiveness of producing only non-transgenic males by the mating scheme in FIG. 1C followed by batch cultivation was tested. A true-breeding culture of DmY^{L-tTA} was transferred to media lacking tetracycline and cleared all adults after 24 hours. The resulting offspring from the tetracycline-free medium were mixed at a 2:1 ratio with adults from a true-breeding population of DmX^{L-tTA} flies. Adults from this cross were cleared after three days. This mating yielded 2932 males (N=3, 977±144) and one female. None of the more than 1000 males screened contained the GFP transgene marker. To ensure the lack of GFP detection (FIG. 4B) was not due to transgene silencing, genomic DNA from was isolated more than 1000 male STSS flies and screened for presence of the transgene by PCR (expected fragment size of 821 bp). A clear band of the expected size from a positive control (gDNA isolated from 10 DmXL^{tTA} flies) but not in gDNA isolated from the putative non-transgenic males (FIG. 4C). Spiking trace amounts of positive control gDNA confirmed that limit of detection via this assay at less than 1:3000 transgenic:non-transgenic gDNA. This confirmed that the assay was sufficiently powerful to detect any transgenic flies that would have been present in the screened population.

This disclosure therefore describes a method of Subtractive Transgene Sex Sorting (STSS) using D. melanogaster as a model system. While described in detail in the context of an exemplary embodiment in which species is D. melanogaster, the methods described herein can involve any organism that relies on genetic, as opposed to environmental, sex determination. Exemplary other species in which the method may be employed include, for example, an insect (e.g., mosquito, tstetse fly, spotted-wing drosophila, diamond back moth, fall army worm, soybean gall midge, white fly, Mediterranean fruit fly, olive fly, gypsy moth, codling moth, deer tick, etc.), a fish (e.g., salmon, carp, sea lamprey, etc.), a bird (e.g., poultry), a mammal (e.g., swine, a mouse, a rat, etc.), an amphibian (e.g., a cane toad, a bullfrog, etc.), a reptile (e.g., brown tree snake, etc.), or a crustacean (e.g., rusty crayfish, etc.).

The method involves the use of two genetically-engineered strains of a pest species and a mating protocol that produces only non-transgenic males. The males may be subsequently sterilized using, for example, SIT sterilization techniques to produce sterile non-transgenic males. The sterile non-transgenic males may be released to control the population of the pest species.

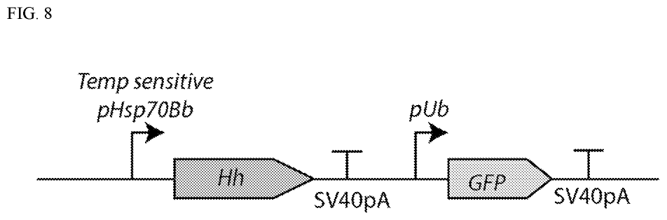

Each strain is engineered to possess a lethal genetic circuit that can be induced or repressed. While described herein in the context of an exemplary embodiment in which both genetically engineered strains possess a tet-transactivator (tTA) genetic circuit that is lethal in the absence of tetracycline, one or both strains may be constructed to include an alternate lethal genetic circuit (e.g. temperature inducible activation of a gene causing lethality, FIG. 8), lactose repressor, methionine repressor). The lethal genetic circuit used in one strain may be independent of—i.e., the same or different than—the lethal genetic circuit used in the other strain.

The basic genetic architecture has been demonstrated in numerous pest insects and STSS can be readily adapted to improve SIT programs by enabling efficient sex-sorting for male only release. Although described in the context of using PhiC31-mediated integration, CRISPR systems also may be used to target integration to many genomic loci in insects, including the repeat rich Y chromosome. This approach allows one to generate transgene-free males in species where males are heterogametic. For species where the female is heterogametic (i.e., lepidoptera), generation of transgene-free males is simplified and would only require a W′ construct. This approach can be applied to species with homomorphic sex chromosomes, assuming the sex-determining region of the sex chromosome is accessible to transgene integration technology.

Using the STSS system, no transgenic flies survives in the absence of tetracycline. The scale of the experiments described herein support a conclusion that the transgenic fly escape is less than 0.1%. SIT programs generate millions of flies for release on a weekly basis and some small number of transgenic flies may be produced and released using the STSS system, although they are likely to suffer fitness defects.

Production of males incapable of reproduction with wild females does not necessarily require radiation treatment. The incompatible insect technique relies on males infected with certain strains of Wolbachia bacteria. Mating between infected males and uninfected females results in embryonic lethality since the female-produced egg does not contain an antidote to a deubiquitylating enzyme toxin delivered by the sperm. Transgenic approaches have been developed or proposed to generate males that cannot reproduce with wild females. So far only Release of Insects with Dominant Lethal (RIDL) has made it to small scale commercial use for the control of Ae. aegypti. RIDL utilizes a bi-sex lethal genetic circuit where larvae, but not adults, require tetracycline to survive to adulthood. Mechanically sorted male pupae are subsequently used for release and their offspring die in the absence of tetracycline in the wild.

Combining STSS with cytoplasmic incompatibility can eliminate the need for any additional sterilization treatments and could enable the release of eggs/larvae to further reduce costs. Shipping of eggs, particularly of species such as Ae. aegypti that can be stored dry for extended periods, would allow for rearing facilities to be located far from control sites and reduce local infrastructure costs. However, this would require using non-antibiotic control of a lethal circuit such as temperature inducible activation of a lethal gene.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Plasmid Construction—Repressible Lethal

The tetracycline repressible lethal circuit was made by adapting a previously described female-lethal piggybac vector, pB[FL3] (Yan et al., 2017, Sci Rep 7:2538). The female specific intron and 5′UTR were replaced with a myosin heavy chain intron and syn21 translational enhancers (Pfeiffer et al., 2012, Proc Natl Acad Sci USA 109: 6626-6631). The final plasmid, pMM7-10-1 (SEQ ID NO:1), was made by transferring the lethal circuit to pUB-EGFP (Schetelig et al., 2009, Proc Natl Acad Sci USA 106: 18171-18176), which contains an attB site for PhiC31 mediated integration and ubiquitin promoter driven EGFP expression.

### Plasmid Construction—Female Lethal

The tetracycline female lethal circuit was made by adapting a previously described female-lethal piggybac vector, pB[FL3] (Li et. al., 2014. Insect Biochem & Mol Biol, 51:80-88). The final plasmid, pMM7-8-1 (SEQ ID NO:2), was made by transferring the lethal circuit to pUB-EGFP (Schetelig et al., 2009, Proc Natl Acad Sci USA 106:18171-18176), which contains an attB site for PhiC31 mediated integration and ubiquitin promoter driven EGFP expression.

### Generating and Maintaining Transgenic Drosophila Strains

D. melanogaster strains were maintained at 25° C. and 12 hours light in cornmeal agar (FLYSTUFF, Genesee Scientific Corp., San Diego, Calif.) supplemented with 10-200 μg/ml tetracycline, as necessary. Transgenic D. melanogaster strains where generated by microinjection (BestGene Inc, Chino Hills, Calif.) and PhiC31 mediated integration of pMM7-10-1 into the X-chromosome attP site of y[1] w[*] P{y[+t7.7]=CaryIP}su(Hw)attP8 (BDSC #3233; Pfeiffer et al., 2010, Genetics 186: 735-755) to make DmXL^{tTA} and the Y-chromosome attP of y1 w*/Dp(2;Y)G, P{CaryP}attPY (Szabad et al., 2012, Genes/Genomes/Genetics 2: 1095-1102) to make DmYL^{tTA}.

### Fly Viability Assays

Desired number of male and virgin female flies were moved to new tubes containing media either with or without tetracycline and allowed to lay eggs for five days at 25° C. and 12 hours light protocol. After five days, adults were removed from the tubes and offspring were allowed to develop in the incubator. Adult flies were counted as they emerged from the pupae for a total of 15 days from the start of experiment.

### PCR Verification

Fly genomic DNA was isolated in a pool by grinding in 25 μl of “Squish Buffer” (10 mM Tris, 1 mM EDTA, 25 mM NaCl, 8 U/ml ProK (New England Biolabs, Inc., Ipswich, Mass.) per adult. ProK was heat inactivated at 98° C. for four minutes. For transgene PCR screen, 1181 STSS males were pooled together as one sample and compared to five male and five female DMX^{L-tTA} flies in a separate pool of genomic DNA as positive control. The positive control samples were diluted in with STSS gDNA in the following ratios: 1:5, 1:25, 1:125, 1:625, and 1:3125. For each reaction, 1 μl, of template gDNA was used in a 20 μL PCR reaction with primers that anneal within the transgene. The following primers were used for amplification of the transgene,

| (SEQ ID NO: 3) | |
|---|---|
| fwd: | 5′-GCCGCAGAATTCTCTCTATC-3′, |
| (SEQ ID NO: 4) | |
| rev: | 5′-CTTAGCTTTCGCTTAGCGACG-3′; |
| (SEQ ID NO: 5) | |
| upd1, fwd: | 5′-TGCAGGTGACCTGGGAATAG-3′, |
| (SEQ ID NO: 6) | |
| upd1, rev: | 5′-GTGAGACCACTTGACCACAG-3′, |
| (SEQ ID NO: 7) | |
| k1-5, fwd: | 5′-CGCGACGATAGACAGCGG-3′, |
| and | |
| (SEQ ID NO: 8) | |
| k1-5, rev: | 5′-GAGAGCAATGCGCTCGTTGC-3′. |

### Data Analysis

All the experiments were performed with at least two and as high as 10 replicates. Raw offspring numbers from each experiment were converted into percent male/female and averaged across the replicates. Chi-squared test was performed to test difference between observed and expected sex ratio in different mating. Number of flies from each experiment across the different replicates was summed together then converted into percent male/female and used in the Chi-squared test. To test the effect of different parental male-female ratio on adult offspring numbers, One-way ANOVA was performed followed by Bonferroni's post-hoc test. P-value <0.05 was considered significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### Sequence Listing Free Text

GGCCCGGTAC GTACCCAATT CGCCCTATAG TGAGTCGTAT TACAATTCAC TGGCCGTCGT TTTACAACGT
CGTGACTGGG AAAACCCTGG CGTTACCCAA CTTAATCGCC TTGCAGCACA TCCCCCTTTC GCCAGCTGGC
GTAATAGCGA AGAGGCCCGC ACCGATCGCC CTTCCCAACA GTTGCGCAGC CTGAATGGCG AATGGAAATT
GTAAGCGTTA ATATTTTGTT AAAATTCGCG TTAAATTTTT GTTAAATCAG CTCATTTTTT AACCAATAGG
CCGAAATCGG CAAAATCCCT TATAAATCAA AAGAATAGAC CGAGATAGGG TTGAGTGTTG TTCCAGTTTG
GAACAAGAGT CCACTATTAA AGAACGTGGA CTCCAACGTC AAAGGGCGAA AAACCGTCTA TCAGGGCGAT
GGCCCACTAC GTGAACCATC ACCCTAATCA AGTTTTTTGG GGTCGAGGTG CCGTAAAGCA CTAAATCGGA
ACCCTAAAGG GAGCCCCCGA TTTAGAGCTT GACGGGGAAA GCCGGCGAAC GTGGCGAGAA AGGAAGGGAA
GAAAGCGAAA GGAGCGGGCG CTAGGGCGCT GGCAAGTGTA GCGGTCACGC TGCGCGTAAC CACCACACCC
GCCGCGCTTA ATGCGCCGCT ACAGGGCGCG TCAGGTGGCA CTTTTCGGGG AAATGTGCGC GGAACCCCTA
TTTGTTTATT TTTCTAAATA CATTCAAATA TGTATCCGCT CATGAGACAA TAACCCTGAT AAATGCTTCA
ATAATATTGA AAAAGGAAGA GTATGAGTAT TCAACATTTC CGTGTCGCCC TTATTCCCTT TTTTGCGGCA
TTTTGCCTTC CTGTTTTTGC TCACCCAGAA ACGCTGGTGA AAGTAAAAGA TGCTGAAGAT CAGTTGGGTG
CACGAGTGGG TTACATCGAA CTGGATCTCA ACAGCGGTAA GATCCTTGAG AGTTTTCGCC CCGAAGAACG
TTTTCCAATG ATGAGCACTT TTAAAGTTCT GCTATGTGGC GCGGTATTAT CCCGTATTGA CGCCGGGCAA
GAGCAACTCG GTCGCCGCAT ACACTATTCT CAGAATGACT TGGTTGAGTA CTCACCAGTC ACAGAAAAGC
ATCTTACGGA TGGCATGACA GTAAGAGAAT TATGCAGTGC TGCCATAACC ATGAGTGATA ACACTGCGGC
CAACTTACTT CTGACAACGA TCGGAGGACC GAAGGAGCTA ACCGCTTTTT TGCACAACAT GGGGGATCAT
GTAACTCGCC TTGATCGTTG GGAACCGGAG CTGAATGAAG CCATACCAAA CGACGAGCGT GACACCACGA
TGCCTGTAGC AATGGCAACA ACGTTGCGCA AACTATTAAC TGGCGAACTA CTTACTCTAG CTTCCCGGCA
ACAATTAATA GACTGGATGG AGGCGGATAA AGTTGCAGGA CCACTTCTGC GCTCGGCCCT TCCGGCTGGC
TGGTTTATTG CTGATAAATC TGGAGCCGGT GAGCGTGGGT CTCGCGGTAT CATTGCAGCA CTGGGGCCAG
ATGGTAAGCC CTCCCGTATC GTAGTTATCT ACACGACGGG GAGTCAGGCA ACTATGGATG AACGAAATAG
ACAGATCGCT GAGATAGGTG CCTCACTGAT TAAGCATTGG TAACTGTCAG ACCAAGTTTA CTCATATATA
CTTTAGATTG ATTTAAAACT TCATTTTTAA TTTAAAAGGA TCTAGGTGAA GATCCTTTTT GATAATCTCA
TGACCAAAAT CCCTTAACGT GAGTTTTCGT TCCACTGAGC GTCAGACCCC GTAGAAAAGA TCAAAGGATC
TTCTTGAGAT CCTTTTTTTC TGCGCGTAAT CTGCTGCTTG CAAACAAAAA AACCACCGCT ACCAGCGGTG
GTTTGTTTGC CGGATCAAGA GCTACCAACT CTTTTTCCGA AGGTAACTGG CTTCAGCAGA GCGCAGATAC
CAAATACTGT CCTTCTAGTG TAGCCGTAGT TAGGCCACCA CTTCAAGAAC TCTGTAGCAC CGCCTACATA
CCTCGCTCTG CTAATCCTGT TACCAGTGGC TGCTGCCAGT GGCGATAAGT CGTGTCTTAC CGGGTTGGAC
TCAAGACGAT AGTTACCGGA TAAGGCGCAG CGGTCGGGCT GAACGGGGGG TTCGTGCACA CAGCCCAGCT
TGGAGCGAAC GACCTACACC GAACTGAGAT ACCTACAGCG TGAGCTATGA GAAAGCGCCA CGCTTCCCGA
AGGGAGAAAG GCGGACAGGT ATCCGGTAAG CGGCAGGGTC GGAACAGGAG AGCGCACGAG GGAGCTTCCA
GGGGGAAACG CCTGGTATCT TTATAGTCCT GTCGGGTTTC GCCACCTCTG ACTTGAGCGT CGATTTTTGT
GATGCTCGTC AGGGGGGCGG AGCCTATGGA AAAACGCCAG CAACGCGGCC TTTTTACGGT TCCTGGCCTT
TTGCTGGCCT TTTGCTCACA TGTTCTTTCC TGCGTTATCC CCTGATTCTG TGGATAACCG TATTACCGCC
TTTGAGTGAG CTGATACCGC TCGCCGCAGC CGAACGACCG AGCGCAGCGA GTCAGTGAGC GAGGAAGCGG
AAGAGCGCCC AATACGCAAA CCGCCTCTCC CCGCGCGTTG GCCGATTCAT TAATGCAGCT GGCACGACAG
GTTTCCCGAC TGGAAAGCGG GCAGTGAGCG CAACGCAATT AATGTGAGTT AGCTCACTCA TTAGGCACCC
CAGGCTTTAC ACTTTATGCT TCCGGCTCGT ATGTTGTGTG GAATTGTGAG CGGATAACAA TTTCACACAG
GAAACAGCTA TGACCATGAT TACGCCAAGC TCGAAATTAA CCCTCACTAA AGGGAACAAA AGCTGGCTAG
AACTAGTGTC GACATGCCCG CCGTGACCGT CGAGAACCCG CTGACGCTGC CCCGCGTATC CGCACCCGCC
GACGCCGTCG CACGTCCCGT GCTCACCGTG ACCACCGCGC CCAGCGGTTT CGAGGGCGAG GGCTTCCCGG
TGCGCCGCGC GTTCGCCGGG ATCAACTACC GCCACCTCGA CCCGTTCATC ATGATGGACC AGATGGGTGA
GGTGGAGTAC GCGCCCGGGG AGCCCAAGGG CACGCCCTGG CACCCGCACC GCGGCTTCGA GACCGTGACC
TACATCGTCG ACACTAGTGG ATCCAGCGGC CGCACCTGCA GGCCGGCCGT TAACACGCGT CCGCGGTCTA
GACTCGAGGA TTCCAGATCT GGTACCGGGC CGCTGTATGG ATATTTGCAG GGCCGCAGAA TTCTCTCTAT
CACTGATAGG GAGGTCTCTA TCACTGATAG GGAGTTCTCT ATCACTGATA GGGATGTCTC TATCACTGAT
AGGGATTTCT CTATCACTGA TAGGGAAGTC TCTATCACTG ATAGGGACCT CTCTATCACT GATAGGGAAA
TCTCTATCAC TGATAGGGAT CTCTCTATCA CTGATAGGGA CTTCTCTATC ACTGATAGGG ACGTCTCTAT
CACTGATAGG GAACTCTCTA TCACTGATAG GGACATCTCT ATCACTGATA GGGACTTCTC TATCACTGAT
AGGGAAGTAT GTTCTCTCTC TTCTCTTCTC TCTCTCTTTC TCGAATGTTC TCTCTCTTCT CTTCTCTCTC
TCTTTCTCGA TGGCCGGGGC GCGCCAGGTT TCGACTTTCA CTTTTCTCTA TCACTGATAG GGAGTGGTAA
ACTCGACTTT CACTTTTCTC TATCACTGAT AGGGAGTGGT AAACTCGACT TTCACTTTTC TCTATCACTG
ATAGGGAGTG GTAAACTCGA CTTTCACTTT TCTCTATCAC TGATAGGGAG ATCCGAGCTC GTAAACTCGA
CTTTCACTTT TCTCTATCAC TGATAGGGAG TGGTAAACTC GACTTTCACT TTTCTCTATC ACTGATAGGG
AGTGGTAAAC TCGACTTTCA CTTTTCTCTA TCACTGATAG GGAGTGGTAA ACTCGAAgcg cCGGATCCGT
CGAGGGAAAA GAGCGCCGGA GTATAAATAG AGGCGCTTCG TCTACGGAGC GACAATTCAA TTCAAACAAG
CAAAGTGAAC ACGTCGCTAA GCGAAAGCTA AGCAAATAAA CAAGCGCAGC TGAACAAGCT AAACAATCTG
CAGCCgatct aaaaggtagg ttcaaccact gatgcctagg cacaccgaaa cgactaaccc taattcttat
cctttacttc aggcggccgg gctcgagggt accaacttaa aaaaaaaaat caaaATGGTC AGCCGTTTGG
ATAAATCCAA AGTTATTAAT TCCGCTTTGG AATTGTTGAA TGAAGTTGGT ATTGAAGGTT TGACAACACG
TAAATTGGCT CAAAAATTGG GTGTTGAACA ACCAACATTG TATTGGCATG TTAAAAATAA ACGTGCTTTG
TTGGATGCTT TGGCTATTGA AATGTTGGAC CGTCATCATA CACATTTTTG CCCATTGGAA GGCGAATCCT
GGCAAGATTT CTTGCGTAAT AATGCCAAAT CCTTCCGTTG TGCTTTGTTG TCCCATCGTG ATGGTGCCAA
GGTTCATTTG GGCACACGTC CAACAGAAAA ACAATATGAA ACATTGGAAA ATCAATTGGC TTTCTTGTGT
CAACAAGGCT TCAGCTTGGA AAATGCTTTG TATGCTTTGA GCGCCGTTGG TCATTTTACA TTGGGCTGTG
TGTTGGAAGA TCAAGAACAT CAAGTCGCTA AAGAAGAACG TGAAACACCA ACAACAGATT CGATGCCCCC
ATTGTTGCGT CAAGCAATTG AATTGTTCGA TCATCAAGGA GCCGAACCAG CATTCTTGTT CGGCTTGGAA
TTGATTATTT GTGGATTGGA AAAACAATTG AAATGTGAAT CGGGCTCGGG CCCCGCGTAC AGCCGCGCGC
GTACGAAAAA CAATTACGGG TCTACCATCG AGGGCCTGCT CGATCTCCCG GACGACGACG CCCCCGAAGA
GGCGGGGCTG GCGGCTCCGC GCCTGTCCTT TCTCCCCGCG GGACACACGC GCAGACTGTC GACGGCCCCC
CCGACCGATG TCAGCCTGGG GGACGAGCTC CACTTAGACG GCGAGGACGT GGCGATGGCG CATGCCGACG
CGCTAGACGA TTTCGATCTG GACATGTTGG GGGACGGGGA TTCCCCGGGT CCGGGATTTA CCCCCCACGA
CTCCGCCCCC TACGGCGCTC TGGATATGGC CGACTTCGAG TTTGAGCAGA TGTTTACCGA TGCCCTTGGA
ATTGACGAGT ACGGTGGGTA GTAAGCTTGG ATCTTTGTGA AGGAACCTTA CTTCTGTGGT GTGACATAAT
TGGACAAACT ACCTACAGAG ATTTAAAGCT CTAAGGTAAA TATAAAATTT TTAAGTGTAT AATGTGTTAA
ACTACTGATT CTAATTGTTT GTGTATTTTA GATTCCAACC TATGGAACTG ATGAATGGGA GCAGTGGTGG
AATGCCTTTA ATGAGGAAAA CCTGTTTTGC TCAGAAGAAA TGCCATCTAG TGATGATGAG GCTACTGCTG
ACTCTCAACA TTCTACTCCT CCAAAAAAGA AGAGAAAGGT AGAAGACCCC AAGGACTTTC CTTCAGAATT
GCTAAGTTTT TTGAGTCATG CTGTGTTTAG TAATAGAACT CTTGCTTGCT TTGCTATTTA CACCACAAAG
GAAAAAGCTG CACTGCTATA CAAGAAAATT ATGGAAAAAT ATTCTGTAAC CTTTATAAGT AGGCATAACA
GTTATAATCA TAACATACTG TTTTTTCTTA CTCCACACAG GCATAGAGTG TCTGCTATTA ATAACTATGC
TCAAAAATTG TGTACCTTTA GCTTTTTAAT TTGTAAAGGG GTTAATAAGG AATATTTGAT GTATAGTGCC
TTGACTAGAG ATCATAATCA GCCATACCAC ATTTGTAGAG GTTTTACTTG CTTTAAAAAA CCTCCCACAC
CTCCCCCTGA ACCTGAAACA TAAAATGAAT GCAATTGTTG TTGTTAACTT GTTTATTGCA GCTTATAATG
GTTACAAATA AAGCAATAGC ATCACAAATT TCACAAATAA AGCATTTTTT TCACTGCATT CTAGTTGTGG
TTTGTCCAAA CTCATCAATG TATCTTATCA TGTCTCGAGC ATGCGCAAAT TTAAAGCGCT GATATCGATC
GCGCGCAGAT CTGTCATGAT GATCATTGCA ATTCTGCAGT CGACGGTACC CGATCTTGTC GCCGGAACGC
AGCGACAGAG ATTCCAATGT GTCCGTATCT TTCAGGCTTT TGCCCTTCAG TTCCAGACGA AGCGACTGGC
GATTCGCGTG TGGGGTCTGC TTCAGGGTCT TGTGAATTAG GGCGCGCAGA TCGCCGATGG GCGTGGCGCC
GGAGGGCACC TTCACCTTGC CGTACGGCTT GCTGTTCTTC GCGTTCAAAA TCTCCAGCTC CATTTTGCTT
TCGGTGCGCT TGCAATCAGT ACTGTCCAAA ATCGAAAATC GCCGAACCGT AGTGTGACCG TGCGGGGCTC
TGCGAAAATA AACTTTTTTA GGTATATGGC CACACACGGG GAAAGCACAG TGGATTATAT GTTTTAATAT
TATAATATGC AGGTTTTCAT TACTTATCCA GATGTAAGCC CACTTAAAGC GATTTAACAA TTATTTGCCG
AAAGAGTAAA AACAAATTTC ACTTAAAAAT GGATTAAGAA AAGCTTGTGT AAGATTATGC GCAGCGTTGC
CAGATAGCTC CATTTAAAAC ACTTCAAAAA CAATAAGTTT TGAAAATATA TACATAAATA GCAGTCGTTG
CCGCAACGCT CAACACATCA CACTTTTAAA ACACCCTTTA CCTACACAGA ATTACTTTTT AAATTTCCAG
TCAAGCTGCG AGTTTCAAAA TTATAGCCGG TAGAGAAGAC AGTGCTATTT CAAAAGCAAA CTAAATAAAC
ACCAATCCTA ACAAGCCTTG GACTTTTGTA AGTTTAGATC AAAGGTGGCA TTGCATTCAA TGTCATGGTA
AGAAGTAGGT CGTCTAGGTA GAAATCCTCA TTCAGCCGGT CAAGTCAGTA CGAGAAAGGT CTCAATTTGA
AATTGTCTTA AAAATATTTT ATTGTTTTGT ACTGTGGTGA GTTTAAACGA AAAACACAAA AAAAAAGTGA
TACACAGAAA TCATAAAAAA TTTTAATACA AGGTATTCGT ACGTATCAAA AACATTTCGG CACAATTTTT
TTTCTCTGTA CTAAAGTGTT ACGAACACTA CGGTATTTTT TAGTGATTTT CAACGGACAC CGAAGGTATA
TAAACAGCGT TCGCGAACGG TCGCCTTCAA AACCAATTGA CATTTGCAGC AGCAAGTACA AGCAGAAAGT
AAAGCGCAAT CAGCGAAAAA TTTATACTTA ATTGTTGGTG ATTAAAGTAC AATTAAAAGA ACATTCTCGA
AAGTCACAAG AAACGTAAGT TTTTAACTCG CTGTTACCAA TTAGTAATAA GAGCAACAAG ACGTTGAGTA
ATTTCAAGAA AAACTGCATT TCAAGGTCTT TGTTCGGCCA TTTTTTTTTT ATTCAACGCT CTACGTAATT
ACAAAATAAG AAATTGGCAG CCACGCATCT TGTTTTCCCA ATCAATTGGC ATCAAAACGC AAACAAATCT
ATAAATAAAA CTTGCGTGTT GATTTTCGCC AAGATTTATT GGCAAATTGT GAAATTCGCA GTGACGCATT
TGAAAATTCG AGAAATCACG AACGCACTCG AGCATTTGTG TGCATGTTAT TAGTTAGTTA GTTCTTTGCT
TAATTGAAGT ATTTTACCAA CGAAATCCAC TTATTTTTAG CTGAAATAGA GTAGGTTGCT TGAAACGAAA
GCCACGTCTG GAAAATTTCT TATTGCTTAG TAGTTGTGAC GTCACCATAT ACACACAAAA TAATGTGTAT
GCATGCGTTT CAGCTGTGTA TATATACATG CACACACTCG CATTATGAAA ACGATGACGA GCAACGGAAC
AGGTTTCTCA ACTACCTTTG TTCCTGTTTC TTCGCTTTCC TTTGTTCCAA TATTCGTAGA GGGTTAATAG
GGGTTTCTCA ACAAAGTTGG CGTCGATAAA TAAGTTTCCC ATTTTTATTC CCCAGCCAGG AAGTTAGTTT
CAATAGTTTT GTAATTTCAA CGAAACTCAT TTGATTTCGT ACTAATTTTC CACATCTCTA TTTTGACCCG
CAGAATAATC CAAAATGCAG ATCGGGGATC CCACCCCACC CAAGAAGAAG CGCAAGGTGG AGGACGATCC
CGTCGTTTTA CAACGTCGTG ACTGGGAAAA CCCTGGCGTT ACCCAACTTA ATCGCCTTGC AGCACATCCC
CCTTTCGCCA GCTGGCGTAA TAGCGAAGAG GCCCGCACCG ATCGCCCTTC CCAACAGTTG CGGTCGACTC
TAGAGGATCC CCGGGATCCA CCGGTCGCCA CCATGGTGAG CAAGGGCGAG GAGCTGTTCA CCGGGGTGGT
GCCCATCCTG GTCGAGCTGG ACGGCGACGT AAACGGCCAC AAGTTCAGCG TGTCCGGCGA GGGCGAGGGC
GATGCCACCT ACGGCAAGCT GACCCTGAAG TTCATCTGCA CCACCGGCAA GCTGCCCGTG CCCTGGCCCA
CCCTCGTGAC CACCCTGACC TACGGCGTGC AGTGCTTCAG CCGCTACCCC GACCACATGA AGCAGCACGA
CTTCTTCAAG TCCGCCATGC CCGAAGGCTA CGTCCAGGAG CGCACCATCT TCTTCAAGGA CGACGGCAAC
TACAAGACCC GCGCCGAGGT GAAGTTCGAG GGCGACACCC TGGTGAACCG CATCGAGCTG AAGGGCATCG
ACTTCAAGGA GGACGGCAAC ATCCTGGGGC ACAAGCTGGA GTACAACTAC AACAGCCACA ACGTCTATAT
CATGGCCGAC AAGCAGAAGA ACGGCATCAA GGTGAACTTC AAGATCCGCC ACAACATCGA GGACGGCAGC
GTGCAGCTCG CCGACCACTA CCAGCAGAAC ACCCCCATCG GCGACGGCCC CGTGCTGCTG CCCGACAACC
ACTACCTGAG CACCCAGTCC GCCCTGAGCA AAGACCCCAA CGAGAAGCGC GATCACATGG TCCTGCTGGA
GTTCGTGACC GCCGCCGGGA TCACTCTCGG CATGGACGAG CTGTACAAGT AAAGCGGCCG CGACTCTAGA
TCATAATCAG CCATACCACA TTTGTAGAGG TTTTACTTGC TTTAAAAAAC CTCCCACACC TCCCCCTGAA
CCTGAAACAT AAAATGAATG CAATTGTTGT TGTTAACTTG TTTATTGCAG CTTATAATGG TTACAAATAA
AGCAATAGCA TCACAAATTT CACAAATAAA GCATTTTTTT CACTGCATTC TAGTTGTGGT TTGTCCAAAC
TCATCAATGT ATCTTAAAGC TTATCGATAC GCGTACGGCA CTAGAGCGGC CGCCACCGCG GTGGAGCTCC
AGCTTTTGTT CCCTTTAGTG AGGGTTAATT AGATCGGCCG GCCTTGGCGC GCCTAGATCT TAATACGACT
CACTATAGGG CGAATTGGGT ACCG

| pMM7-8-1-PUbEGFP, 12394 bp ds-DNA | |
|---|---|
| SEQ ID NO: 2 | |
| 1 | GGCCCGGTAC GTACCCAATT CGCCCTATAG TGAGTCGTAT TACAATTCAC TGGCCGTCGT |
| 61 | TTTACAACGT CGTGACTGGG AAAACCCTGG CGTTACCCAA CTTAATCGCC TTGCAGCACA |
| 121 | TCCCCCTTTC GCCAGCTGGC GTAATAGCGA AGAGGCCCGC ACCGATCGCC CTTCCCAACA |
| 181 | GTTGCGCAGC CTGAATGGCG AATGGAAATT GTAAGCGTTA ATATTTTGTT AAAATTCGCG |
| 241 | TTAAATTTTT GTTAAATCAG CTCATTTTTT AACCAATAGG CCGAAATCGG CAAAATCCCT |
| 301 | TATAAATCAA AAGAATAGAC CGAGATAGGG TTGAGTGTTG TTCCAGTTTG GAACAAGAGT |
| 361 | CCACTATTAA AGAACGTGGA CTCCAACGTC AAAGGGCGAA AAACCGTCTA TCAGGGCGAT |
| 421 | GGCCCACTAC GTGAACCATC ACCCTAATCA AGTTTTTTGG GGTCGAGGTG CCGTAAAGCA |
| 481 | CTAAATCGGA ACCCTAAAGG GAGCCCCCGA TTTAGAGCTT GACGGGGAAA GCCGGCGAAC |
| 541 | GTGGCGAGAA AGGAAGGGAA GAAAGCGAAA GGAGCGGGCG CTAGGGCGCT GGCAAGTGTA |
| 601 | GCGGTCACGC TGCGCGTAAC CACCACACCC GCCGCGCTTA ATGCGCCGCT ACAGGGCGCG |
| 661 | TCAGGTGGCA CTTTTCGGGG AAATGTGCGC GGAACCCCTA TTTGTTTATT TTTCTAAATA |
| 721 | CATTCAAATA TGTATCCGCT CATGAGACAA TAACCCTGAT AAATGCTTCA ATAATATTGA |
| 781 | AAAAGGAAGA GTATGAGTAT TCAACATTTC CGTGTCGCCC TTATTCCCTT TTTTGCGGCA |
| 841 | TTTTGCCTTC CTGTTTTTGC TCACCCAGAA ACGCTGGTGA AAGTAAAAGA TGCTGAAGAT |
| 901 | CAGTTGGGTG CACGAGTGGG TTACATCGAA CTGGATCTCA ACAGCGGTAA GATCCTTGAG |
| 961 | AGTTTTCGCC CCGAAGAACG TTTTCCAATG ATGAGCACTT TTAAAGTTCT GCTATGTGGC |
| 1021 | GCGGTATTAT CCCGTATTGA CGCCGGGCAA GAGCAACTCG GTCGCCGCAT ACACTATTCT |
| 1081 | CAGAATGACT TGGTTGAGTA CTCACCAGTC ACAGAAAAGC ATCTTACGGA TGGCATGACA |
| 1141 | GTAAGAGAAT TATGCAGTGC TGCCATAACC ATGAGTGATA ACACTGCGGC CAACTTACTT |
| 1201 | CTGACAACGA TCGGAGGACC GAAGGAGCTA ACCGCTTTTT TGCACAACAT GGGGGATCAT |
| 1261 | GTAACTCGCC TTGATCGTTG GGAACCGGAG CTGAATGAAG CCATACCAAA CGACGAGCGT |
| 1321 | GACACCACGA TGCCTGTAGC AATGGCAACA ACGTTGCGCA AACTATTAAC TGGCGAACTA |
| 1381 | CTTACTCTAG CTTCCCGGCA ACAATTAATA GACTGGATGG AGGCGGATAA AGTTGCAGGA |
| 1441 | CCACTTCTGC GCTCGGCCCT TCCGGCTGGC TGGTTTATTG CTGATAAATC TGGAGCCGGT |
| 1501 | GAGCGTGGGT CTCGCGGTAT CATTGCAGCA CTGGGGCCAG ATGGTAAGCC CTCCCGTATC |
| 1561 | GTAGTTATCT ACACGACGGG GAGTCAGGCA ACTATGGATG AACGAAATAG ACAGATCGCT |
| 1621 | GAGATAGGTG CCTCACTGAT TAAGCATTGG TAACTGTCAG ACCAAGTTTA CTCATATATA |
| 1681 | CTTTAGATTG ATTTAAAACT TCATTTTTAA TTTAAAAGGA TCTAGGTGAA GATCCTTTTT |
| 1741 | GATAATCTCA TGACCAAAAT CCCTTAACGT GAGTTTTCGT TCCACTGAGC GTCAGACCCC |
| 1801 | GTAGAAAAGA TCAAAGGATC TTCTTGAGAT CCTTTTTTTC TGCGCGTAAT CTGCTGCTTG |
| 1861 | CAAACAAAAA AACCACCGCT ACCAGCGGTG GTTTGTTTGC CGGATCAAGA GCTACCAACT |
| 1921 | CTTTTTCCGA AGGTAACTGG CTTCAGCAGA GCGCAGATAC CAAATACTGT CCTTCTAGTG |
| 1981 | TAGCCGTAGT TAGGCCACCA CTTCAAGAAC TCTGTAGCAC CGCCTACATA CCTCGCTCTG |
| 2041 | CTAATCCTGT TACCAGTGGC TGCTGCCAGT GGCGATAAGT CGTGTCTTAC CGGGTTGGAC |
| 2101 | TCAAGACGAT AGTTACCGGA TAAGGCGCAG CGGTCGGGCT GAACGGGGGG TTCGTGCACA |
| 2161 | CAGCCCAGCT TGGAGCGAAC GACCTACACC GAACTGAGAT ACCTACAGCG TGAGCTATGA |
| 2221 | GAAAGCGCCA CGCTTCCCGA AGGGAGAAAG GCGGACAGGT ATCCGGTAAG CGGCAGGGTC |
| 2281 | GGAACAGGAG AGCGCACGAG GGAGCTTCCA GGGGGAAACG CCTGGTATCT TTATAGTCCT |
| 2341 | GTCGGGTTTC GCCACCTCTG ACTTGAGCGT CGATTTTTGT GATGCTCGTC AGGGGGGCGG |
| 2401 | AGCCTATGGA AAAACGCCAG CAACGCGGCC TTTTTACGGT TCCTGGCCTT TTGCTGGCCT |
| 2461 | TTTGCTCACA TGTTCTTTCC TGCGTTATCC CCTGATTCTG TGGATAACCG TATTACCGCC |
| 2521 | TTTGAGTGAG CTGATACCGC TCGCCGCAGC CGAACGACCG AGCGCAGCGA GTCAGTGAGC |
| 2581 | GAGGAAGCGG AAGAGCGCCC AATACGCAAA CCGCCTCTCC CCGCGCGTTG GCCGATTCAT |
| 2641 | TAATGCAGCT GGCACGACAG GTTTCCCGAC TGGAAAGCGG GCAGTGAGCG CAACGCAATT |
| 2701 | AATGTGAGTT AGCTCACTCA TTAGGCACCC CAGGCTTTAC ACTTTATGCT TCCGGCTCGT |
| 2761 | ATGTTGTGTG GAATTGTGAG CGGATAACAA TTTCACACAG GAAACAGCTA TGACCATGAT |
| 2821 | TACGCCAAGC TCGAAATTAA CCCTCACTAA AGGGAACAAA AGCTGGCTAG AACTAGTGTC |
| 2881 | GACATGCCCG CCGTGACCGT CGAGAACCCG CTGACGCTGC CCCGCGTATC CGCACCCGCC |
| 2941 | GACGCCGTCG CACGTCCCGT GCTCACCGTG ACCACCGCGC CCAGCGGTTT CGAGGGCGAG |
| 3001 | GGCTTCCCGG TGCGCCGCGC GTTCGCCGGG ATCAACTACC GCCACCTCGA CCCGTTCATC |
| 3061 | ATGATGGACC AGATGGGTGA GGTGGAGTAC GCGCCCGGGG AGCCCAAGGG CACGCCCTGG |
| 3121 | CACCCGCACC GCGGCTTCGA GACCGTGACC TACATCGTCG ACACTAGTGG ATCCAGCGGC |
| 3181 | CGCACCTGCA GGCCGGCCGT TAACACGCGT CCGCGGTCTA GACTCGAGGA TTCCAGATCT |
| 3241 | GGTACCGGGC CGCTGTATGG ATATTTGCAG GGCCGCAGAA TTCTCTCTAT CACTGATAGG |
| 3301 | GAGGTCTCTA TCACTGATAG GGAGTTCTCT ATCACTGATA GGGATGTCTC TATCACTGAT |
| 3361 | AGGGATTTCT CTATCACTGA TAGGGAAGTC TCTATCACTG ATAGGGACCT CTCTATCACT |
| 3421 | GATAGGGAAA TCTCTATCAC TGATAGGGAT CTCTCTATCA CTGATAGGGA CTTCTCTATC |
| 3481 | ACTGATAGGG ACGTCTCTAT CACTGATAGG GAACTCTCTA TCACTGATAG GGACATCTCT |
| 3541 | ATCACTGATA GGGACTTCTC TATCACTGAT AGGGAAGTAT GTTCTCTCTC TTCTCTTCTC |
| 3601 | TCTCTCTTTC TCGAATGTTC TCTCTCTTCT CTTCTCTCTC TCTTTCTCGA TGGCCGGGGC |
| 3661 | GCGCCAGGTT TCGACTTTCA CTTTTCTCTA TCACTGATAG GGAGTGGTAA ACTCGACTTT |
| 3721 | CACTTTTCTC TATCACTGAT AGGGAGTGGT AAACTCGACT TTCACTTTTC TCTATCACTG |
| 3781 | ATAGGGAGTG GTAAACTCGA CTTTCACTTT TCTCTATCAC TGATAGGGAG ATCCGAGCTC |
| 3841 | GTAAACTCGA CTTTCACTTT TCTCTATCAC TGATAGGGAG TGGTAAACTC GACTTTCACT |
| 3901 | TTTCTCTATC ACTGATAGGG AGTGGTAAAC TCGACTTTCA CTTTTCTCTA TCACTGATAG |
| 3961 | GGAGTGGTAA ACTCGAACGG ATCCGTCGAG GGAAAAGAGC GCCGGAGTAT AAATAGAGGC |
| 4021 | GCTTCGTCTA CGGAGCGACA ATTCAATTCA AACAAGCAAA GTGAACACGT CGCTAAGCGA |
| 4081 | AAGCTAAGCA AATAAACAAG CGCAGCTGAA CAAGCTAAAC AATCTGCAGC CATGGTAATT |
| 4141 | TTCTTTACGT ATATCAAGTG TTACGGCTCC ATTTTTCTTT AGATATTTCC AGTATAGTTT |
| 4201 | TTTATTACCA ACATTTAAAA ACAAATTTTA GAAAGCATAC TGTTGGGATT TAAATGATTT |
| 4261 | TTTTATTAAA AAGTGAGACA AAATTTTCAA TACAGTTTTA ATAATGGCAA AAGAAAATAT |
| 4321 | ACTGAAAACG TTGCATTTTC CAAGAGGAAC AAACTACAAT CAACATACTA TGTCTTGGTT |
| 4381 | TGAAGAAGAA GTTGTGACAT ATTGGGCAGT TAAAACAAGA CTATAACAGT GAGTATTATA |
| 4441 | AAAAAATTGT TAAAATAACA TATTCCTATA TATATTTATA GCATTTTAAA TAAATATTAA |
| 4501 | ACATTTATTT ATCATTAAGT TATAAGACAT ATATTCAAAT ATTGTTGTAA CAGCTGTAAA |
| 4561 | AACAAGTTAG TTAATTGTTA TTATTCAGGT TCTGGTTAAA CTCCAGGTCA TGAAATTGTG |
| 4621 | TCTCTTCTAT AAGATAAGTC CCTGGATCTT CTGGAGTAAG AGTGGTAGGA TTGGCTTATC |
| 4681 | AGTTAAACAA ATGTTCTGTA TCATGTGGTT TGACCACATA CTGGACAAAT ATTAGGGACG |
| 4741 | GCACGGTCAA TACGAGACTA GTATGCATCG AGACTATTGA TCCACCCCAA GCGTAGTTGT |
| 4801 | TCCAGAGCTT CTTTTGTAGA CCTCGGTAGA TTCTGTTCTA TAAAAACCAA GTATCATGCT |
| 4861 | TCTTACTGAT GAGAATTAAC GAGAACGTAA AGCGCCTACA CATGTTTATA ATGTATTCCC |
| 4921 | GCAAGCATAT GTATACATTG TAATCTCTGT GTGACCATTA TATTTATAAT CTTTGATATA |
| 4981 | AACCTTATTC CAGGGTACTA AAGATATATT TTAATTTTAT TTCTTTTGAA TGTCTGTTGC |
| 5041 | AAAATATGTA AATATATAAA AACTTTTATG AAAATATTAT TTAAGTAAAG ATATAAAATA |
| 5101 | GTTGAAAAAA TCTTTGAATT GTAGAAAAAT AGTCCGCTCC CCTACTTGAT GCCCATATTC |
| 5161 | GCATTCGGTG TAGTCATCTT GTTCCATGCT TTAAGCCGCT TCTCACAAGT ATTCCAGTAA |
| 5221 | GAGGTCACCC GTGTTAATTA GGCATAGCTA TCAGTTTATT TTAGCACTAT TTGATCATCT |
| 5281 | AAATCCCTGC TCCTGCTAAC TACCACCTTG TACTTAAAGT ACATAAAATT TGGTCTTCAG |
| 5341 | CATCGTTCTT CGAGGGGCGG TCATAATATT TTTTATATTT TAAGGAGTGA GATCGAACGT |
| 5401 | TTTTAAAGTG CTGTAATTTT GCTCGAATAG GTAGTACATC TCGTTTTAAA ATCTAACACT |
| 5461 | TGGAAACCTA TTTTGTGCCC TTCAATTAAT AATTATCATG AGCATAATAT CTCTCTACTG |
| 5521 | GCCTTAGCTC CGGGCTTTTT GGAGAAAAAA AGTCGGCACA TAATGAAGTC TTATAAATGA |
| 5581 | AAACAGTCTT TCTTGTAAAC GTTCCTTGAT TTATATTTAT AGAGCCTGTT GTAACAAATA |
| 5641 | ATTAGCTTCT TAAAAAGAAC TTGACTGATT TTGGGTCCTA AATTTTTCTT CGACATTCTC |
| 5701 | TTGAGCATCA GCAACATAAA ATTTTTTATT AGGTAGTTGC AAAAAAACTG CCATGATTAG |
| 5761 | TCATCCATTA TGCAACAAGA ATACTTTTTC AAGAAAACTT GATTTTAATT TCCGTACTCT |
| 5821 | GTTTTAGGCC TCTAATTTTT TGAGCAACAT TCCTGTCAAA AACTTTTTGA GCCTTGTACG |
| 5881 | TTTTTAAACC TGCATCAGCT TTAACTTTTC GTACCAAATA GTCCGACCAA TGAGCTAACC |
| 5941 | GGGCTTTTCT ACCTAATATG TTGGCAGCTC TTTTGAAAAT ATTTTGAGAC ATCATGTGAA |
| 6001 | CCATTGCTTC CACATGAACC AGTTTTCTCT GGGTACTGTT TGAAAAACAT AGGAAACAGC |
| 6061 | TTGGCGGCAA ACCTTTGAAT GCTTGGGCAA CTTTTGTGGG ACAAAGTTTT GGTTTGTTGA |
| 6121 | AAATATTTAA TAATTTATTA GGCACTTTTT TCTGGTCCCT CATTTAAATC GGATTACCAA |
| 6181 | TTTCATTTGT TTTTAATTAA ACATTATAAA TTATATGTTT TACATGTTTC ACTAAACGTG |
| 6241 | TGTTACTAAT TTTTCGATCT CACTCCTTTT ATACCGTATT ATATTAATTC TATACTGTAA |
| 6301 | TTAAAGTTAT TTTCAAATTG TTGCAATATT TATTTAGCAA AATGTTTTCA TAACGTGAAA |
| 6361 | TTGTATGTTA TTTTTAGAAA AAATGTATAT TAAGTTCTCA AATTCATATT GTTTATTCAT |
| 6421 | TTAGATTCCC TTGAACAAAA GGGGTTTGGT ATTTTGTAAA AAATTACTAT ATTATTTAAT |
| 6481 | AAGTAGTTAA GATTAATGAA TTTCAGTGAA TAATAAAAGC TCAACAATCA ACATACTAAC |
| 6541 | ATTTTGAAGA TCAGCAATAT TAATCTATCA ACACTAATTA TAATTAACGA CAATCAACAT |
| 6601 | ACCATAGAAA AAAGGATAAT GGATAATGAA TACAAAACTA CAATCAACAT TTTCCTCAGG |
| 6661 | GCAACACACA TCTAGGTTTT GCAAGGATCA ACAAATCAAG AGTGCATTAA AAATAACAAC |
| 6721 | AATCAACATA CCATAATTGA AGATGTTGCA AATATTGAAA ATTTTTATTA AAAACATTTA |
| 6781 | AAATTTACTT AAATTTTTCC TTTAAACGCA AATAAAAAGA AAACTTAAAT TATTCTATTG |
| 6841 | CAAACAGAAA AAATCCCAAA TTAAAATTTA TTTAAAAATT ATTTTTGTTA TAAAACAAAT |
| 6901 | CTAAAATCTA TTTAATTTTA AAAATAATTA AAAAAAAACA TAAACGTGTT AAAACAATTT |
| 6961 | CACAGCTTAA AAATATCGAT AAAAAATATA TAATTTTTAA TAATTTATTT TAATTAATCA |
| 7021 | TCTTTATCAA CATACAAAAT GATAGATAGA TTTTAAAAGG ATCGAGGTTG CATGTATGAT |
| 7081 | AAATTTATTA TTCTTTTCTA TGTTTAGGTC AGCCGTTTGG ATAAATCCAA AGTTATTAAT |
| 7141 | TCCGCTTTGG AATTGTTGAA TGAAGTTGGT ATTGAAGGTT TGACAACACG TAAATTGGCT |
| 7201 | CAAAAATTGG GTGTTGAACA ACCAACATTG TATTGGCATG TTAAAAATAA ACGTGCTTTG |
| 7261 | TTGGATGCTT TGGCTATTGA AATGTTGGAC CGTCATCATA CACATTTTTG CCCATTGGAA |
| 7321 | GGCGAATCCT GGCAAGATTT CTTGCGTAAT AATGCCAAAT CCTTCCGTTG TGCTTTGTTG |
| 7381 | TCCCATCGTG ATGGTGCCAA GGTTCATTTG GGCACACGTC CAACAGAAAA ACAATATGAA |
| 7441 | ACATTGGAAA ATCAATTGGC TTTCTTGTGT CAACAAGGCT TCAGCTTGGA AAATGCTTTG |
| 7501 | TATGCTTTGA GCGCCGTTGG TCATTTTACA TTGGGCTGTG TGTTGGAAGA TCAAGAACAT |
| 7561 | CAAGTCGCTA AAGAAGAACG TGAAACACCA ACAACAGATT CGATGCCCCC ATTGTTGCGT |
| 7621 | CAAGCAATTG AATTGTTCGA TCATCAAGGA GCCGAACCAG CATTCTTGTT CGGCTTGGAA |
| 7681 | TTGATTATTT GTGGATTGGA AAAACAATTG AAATGTGAAT CGGGCTCGGG CCCCGCGTAC |
| 7741 | AGCCGCGCGC GTACGAAAAA CAATTACGGG TCTACCATCG AGGGCCTGCT CGATCTCCCG |
| 7801 | GACGACGACG CCCCCGAAGA GGCGGGGCTG GCGGCTCCGC GCCTGTCCTT TCTCCCCGCG |
| 7861 | GGACACACGC GCAGACTGTC GACGGCCCCC CCGACCGATG TCAGCCTGGG GGACGAGCTC |
| 7921 | CACTTAGACG GCGAGGACGT GGCGATGGCG CATGCCGACG CGCTAGACGA TTTCGATCTG |
| 7981 | GACATGTTGG GGGACGGGGA TTCCCCGGGT CCGGGATTTA CCCCCCACGA CTCCGCCCCC |
| 8041 | TACGGCGCTC TGGATATGGC CGACTTCGAG TTTGAGCAGA TGTTTACCGA TGCCCTTGGA |
| 8101 | ATTGACGAGT ACGGTGGGTA GTAAGCTTGG ATCTTTGTGA AGGAACCTTA CTTCTGTGGT |
| 8161 | GTGACATAAT TGGACAAACT ACCTACAGAG ATTTAAAGCT CTAAGGTAAA TATAAAATTT |
| 8221 | TTAAGTGTAT AATGTGTTAA ACTACTGATT CTAATTGTTT GTGTATTTTA GATTCCAACC |
| 8281 | TATGGAACTG ATGAATGGGA GCAGTGGTGG AATGCCTTTA ATGAGGAAAA CCTGTTTTGC |
| 8341 | TCAGAAGAAA TGCCATCTAG TGATGATGAG GCTACTGCTG ACTCTCAACA TTCTACTCCT |
| 8401 | CCAAAAAAGA AGAGAAAGGT AGAAGACCCC AAGGACTTTC CTTCAGAATT GCTAAGTTTT |
| 8461 | TTGAGTCATG CTGTGTTTAG TAATAGAACT CTTGCTTGCT TTGCTATTTA CACCACAAAG |
| 8521 | GAAAAAGCTG CACTGCTATA CAAGAAAATT ATGGAAAAAT ATTCTGTAAC CTTTATAAGT |
| 8581 | AGGCATAACA GTTATAATCA TAACATACTG TTTTTTCTTA CTCCACACAG GCATAGAGTG |
| 8641 | TCTGCTATTA ATAACTATGC TCAAAAATTG TGTACCTTTA GCTTTTTAAT TTGTAAAGGG |
| 8701 | GTTAATAAGG AATATTTGAT GTATAGTGCC TTGACTAGAG ATCATAATCA GCCATACCAC |
| 8761 | ATTTGTAGAG GTTTTACTTG CTTTAAAAAA CCTCCCACAC CTCCCCCTGA ACCTGAAACA |
| 8821 | TAAAATGAAT GCAATTGTTG TTGTTAACTT GTTTATTGCA GCTTATAATG GTTACAAATA |
| 8881 | AAGCAATAGC ATCACAAATT TCACAAATAA AGCATTTTTT TCACTGCATT CTAGTTGTGG |
| 8941 | TTTGTCCAAA CTCATCAATG TATCTTATCA TGTCTCGAGC ATGCGCAAAT TTAAAGCGCT |
| 9001 | GATATCGATC GCGCGCAGAT CTGTCATGAT GATCATTGCA ATTCTGCAGT CGACGGTACC |
| 9061 | CGATCTTGTC GCCGGAACGC AGCGACAGAG ATTCCAATGT GTCCGTATCT TTCAGGCTTT |
| 9121 | TGCCCTTCAG TTCCAGACGA AGCGACTGGC GATTCGCGTG TGGGGTCTGC TTCAGGGTCT |
| 9181 | TGTGAATTAG GGCGCGCAGA TCGCCGATGG GCGTGGCGCC GGAGGGCACC TTCACCTTGC |
| 9241 | CGTACGGCTT GCTGTTCTTC GCGTTCAAAA TCTCCAGCTC CATTTTGCTT TCGGTGCGCT |
| 9301 | TGCAATCAGT ACTGTCCAAA ATCGAAAATC GCCGAACCGT AGTGTGACCG TGCGGGGCTC |
| 9361 | TGCGAAAATA AACTTTTTTA GGTATATGGC CACACACGGG GAAAGCACAG TGGATTATAT |
| 9421 | GTTTTAATAT TATAATATGC AGGTTTTCAT TACTTATCCA GATGTAAGCC CACTTAAAGC |
| 9481 | GATTTAACAA TTATTTGCCG AAAGAGTAAA AACAAATTTC ACTTAAAAAT GGATTAAGAA |
| 9541 | AAGCTTGTGT AAGATTATGC GCAGCGTTGC CAGATAGCTC CATTTAAAAC ACTTCAAAAA |
| 9601 | CAATAAGTTT TGAAAATATA TACATAAATA GCAGTCGTTG CCGCAACGCT CAACACATCA |
| 9661 | CACTTTTAAA ACACCCTTTA CCTACACAGA ATTACTTTTT AAATTTCCAG TCAAGCTGCG |
| 9721 | AGTTTCAAAA TTATAGCCGG TAGAGAAGAC AGTGCTATTT CAAAAGCAAA CTAAATAAAC |
| 9781 | ACCAATCCTA ACAAGCCTTG GACTTTTGTA AGTTTAGATC AAAGGTGGCA TTGCATTCAA |
| 9841 | TGTCATGGTA AGAAGTAGGT CGTCTAGGTA GAAATCCTCA TTCAGCCGGT CAAGTCAGTA |
| 9901 | CGAGAAAGGT CTCAATTTGA AATTGTCTTA AAAATATTTT ATTGTTTTGT ACTGTGGTGA |
| 9961 | GTTTAAACGA AAAACACAAA AAAAAAGTGA TACACAGAAA TCATAAAAAA TTTTAATACA |
| 10021 | AGGTATTCGT ACGTATCAAA AACATTTCGG CACAATTTTT TTTCTCTGTA CTAAAGTGTT |
| 10081 | ACGAACACTA CGGTATTTTT TAGTGATTTT CAACGGACAC CGAAGGTATA TAAACAGCGT |
| 10141 | TCGCGAACGG TCGCCTTCAA AACCAATTGA CATTTGCAGC AGCAAGTACA AGCAGAAAGT |
| 10201 | AAAGCGCAAT CAGCGAAAAA TTTATACTTA ATTGTTGGTG ATTAAAGTAC AATTAAAAGA |
| 10261 | ACATTCTCGA AAGTCACAAG AAACGTAAGT TTTTAACTCG CTGTTACCAA TTAGTAATAA |
| 10321 | GAGCAACAAG ACGTTGAGTA ATTTCAAGAA AAACTGCATT TCAAGGTCTT TGTTCGGCCA |
| 10381 | TTTTTTTTTT ATTCAACGCT CTACGTAATT ACAAAATAAG AAATTGGCAG CCACGCATCT |
| 10441 | TGTTTTCCCA ATCAATTGGC ATCAAAACGC AAACAAATCT ATAAATAAAA CTTGCGTGTT |
| 10501 | GATTTTCGCC AAGATTTATT GGCAAATTGT GAAATTCGCA GTGACGCATT TGAAAATTCG |
| 10561 | AGAAATCACG AACGCACTCG AGCATTTGTG TGCATGTTAT TAGTTAGTTA GTTCTTTGCT |
| 10621 | TAATTGAAGT ATTTTACCAA CGAAATCCAC TTATTTTTAG CTGAAATAGA GTAGGTTGCT |
| 10681 | TGAAACGAAA GCCACGTCTG GAAAATTTCT TATTGCTTAG TAGTTGTGAC GTCACCATAT |
| 10741 | ACACACAAAA TAATGTGTAT GCATGCGTTT CAGCTGTGTA TATATACATG CACACACTCG |
| 10801 | CATTATGAAA ACGATGACGA GCAACGGAAC AGGTTTCTCA ACTACCTTTG TTCCTGTTTC |
| 10861 | TTCGCTTTCC TTTGTTCCAA TATTCGTAGA GGGTTAATAG GGGTTTCTCA ACAAAGTTGG |
| 10921 | CGTCGATAAA TAAGTTTCCC ATTTTTATTC CCCAGCCAGG AAGTTAGTTT CAATAGTTTT |
| 10981 | GTAATTTCAA CGAAACTCAT TTGATTTCGT ACTAATTTTC CACATCTCTA TTTTGACCCG |
| 11041 | CAGAATAATC CAAAATGCAG ATCGGGGATC CCACCCCACC CAAGAAGAAG CGCAAGGTGG |
| 11101 | AGGACGATCC CGTCGTTTTA CAACGTCGTG ACTGGGAAAA CCCTGGCGTT ACCCAACTTA |
| 11161 | ATCGCCTTGC AGCACATCCC CCTTTCGCCA GCTGGCGTAA TAGCGAAGAG GCCCGCACCG |
| 11221 | ATCGCCCTTC CCAACAGTTG CGGTCGACTC TAGAGGATCC CCGGGATCCA CCGGTCGCCA |
| 11281 | CCATGGTGAG CAAGGGCGAG GAGCTGTTCA CCGGGGTGGT GCCCATCCTG GTCGAGCTGG |
| 11341 | ACGGCGACGT AAACGGCCAC AAGTTCAGCG TGTCCGGCGA GGGCGAGGGC GATGCCACCT |
| 11401 | ACGGCAAGCT GACCCTGAAG TTCATCTGCA CCACCGGCAA GCTGCCCGTG CCCTGGCCCA |
| 11461 | CCCTCGTGAC CACCCTGACC TACGGCGTGC AGTGCTTCAG CCGCTACCCC GACCACATGA |
| 11521 | AGCAGCACGA CTTCTTCAAG TCCGCCATGC CCGAAGGCTA CGTCCAGGAG CGCACCATCT |
| 11581 | TCTTCAAGGA CGACGGCAAC TACAAGACCC GCGCCGAGGT GAAGTTCGAG GGCGACACCC |
| 11641 | TGGTGAACCG CATCGAGCTG AAGGGCATCG ACTTCAAGGA GGACGGCAAC ATCCTGGGGC |
| 11701 | ACAAGCTGGA GTACAACTAC AACAGCCACA ACGTCTATAT CATGGCCGAC AAGCAGAAGA |
| 11761 | ACGGCATCAA GGTGAACTTC AAGATCCGCC ACAACATCGA GGACGGCAGC GTGCAGCTCG |
| 11821 | CCGACCACTA CCAGCAGAAC ACCCCCATCG GCGACGGCCC CGTGCTGCTG CCCGACAACC |
| 11881 | ACTACCTGAG CACCCAGTCC GCCCTGAGCA AAGACCCCAA CGAGAAGCGC GATCACATGG |
| 11941 | TCCTGCTGGA GTTCGTGACC GCCGCCGGGA TCACTCTCGG CATGGACGAG CTGTACAAGT |
| 12001 | AAAGCGGCCG CGACTCTAGA TCATAATCAG CCATACCACA TTTGTAGAGG TTTTACTTGC |
| 12061 | TTTAAAAAAC CTCCCACACC TCCCCCTGAA CCTGAAACAT AAAATGAATG CAATTGTTGT |
| 12121 | TGTTAACTTG TTTATTGCAG CTTATAATGG TTACAAATAA AGCAATAGCA TCACAAATTT |
| 12181 | CACAAATAAA GCATTTTTTT CACTGCATTC TAGTTGTGGT TTGTCCAAAC TCATCAATGT |
| 12241 | ATCTTAAAGC TTATCGATAC GCGTACGGCA CTAGAGCGGC CGCCACCGCG GTGGAGCTCC |
| 12301 | AGCTTTTGTT CCCTTTAGTG AGGGTTAATT AGATCGGCCG GCCTTGGCGC GCCTAGATCT |
| 12361 | TAATACGACT CACTATAGGG CGAATTGGGT ACCG |

primer
SEQ ID NO: 3
GCCGCAGAAT TCTCTCTATC
primer
SEQ ID NO: 4
CTTAGCTTTC GCTTAGCGAC G
primer
SEQ ID NO: 5
TGCAGGTGAC CTGGGAATAG
primer
SEQ ID NO: 6
GTGAGACCAC TTGACCACAG
primer
SEQ ID NO: 7
CGCGACGATA GACAGCGG
primer
SEQ ID NO: 8
GAGAGCAATG CGCTCGTTGC

1. A system comprising:
a first strain of a biological species genetically engineered to comprise a conditional P-linked genetic lethal circuit; and
a second strain of the biological species genetically engineered to comprise a conditional X-linked genetic lethal circuit.

2. The system of claim 1, wherein the X-linked genetic lethal circuit is the same as the Y-linked genetic lethal circuit.

3. The system of claim 1, wherein the X-linked genetic lethal circuit is different than the Y-linked genetic lethal circuit.

4. The system of claim 1, wherein the biological species is a pest species.

5. The system of claim 1, wherein the biological species is a species in which one sex has greater commercial value than the other sex.

6. The system of claim 1, wherein the conditional X-linked genetic lethal circuit is lethal only to females under conditions effective to express the conditional X-linked lethal circuit.

7. A method of selecting non-transgenic males of a biological species, the method comprising:
providing a first strain of the biological species genetically engineered to comprise a conditional Y-linked genetic lethal circuit;
providing a second strain of the biological species genetically engineered to comprise a conditional X-linked genetic lethal circuit;
performing a first cross mating males of the first strain and females of the first strain under conditions effective to express the conditional Y-linked genetic lethal circuit, thereby producing non-transgenic female progeny; and
mating the non-transgenic progeny of the first cross with males of the second strain under conditions effective to express the conditional X-linked genetic lethal circuit, thereby producing non-transgenic males.

8. The method of claim 7, wherein the X-linked genetic lethal circuit is the same as the Y-linked genetic lethal circuit.

9. The method of claim 7, wherein the X-linked genetic lethal circuit is different than the Y-linked genetic lethal circuit.

10. The system of claim 7, wherein the biological species is a pest species.

11. The system of claim 7, wherein the biological species is a species in which one sex has greater commercial value than the other sex.

12. A method of producing a population of sterile non-transgenic males of a biological species, the method comprising:
providing the non-transgenic males selected using the method of claim 7; and
subjecting the non-transgenic males to a treatment effective to sterilize the males.

13. The method of claim 12, wherein the males are sterilized by subjecting the males to X-ray irradiation.

14. The method of claim 12, wherein the biological species is a pest species.

15. A system comprising:
a first strain of a biological species genetically engineered to comprise a conditional W-linked genetic lethal circuit; and
a second strain of the biological species genetically engineered to comprise a conditional Z-linked genetic lethal circuit.

16. The system of claim 15, wherein the Z-linked genetic lethal circuit is the same as the W-linked genetic lethal circuit.

17. The system of claim 15, wherein the Z-linked genetic lethal circuit is different than the W-linked genetic lethal circuit.

18. The system of claim 15, wherein the biological species is a pest species.

19. The system of claim 15, wherein the biological species is a species in which one sex has greater commercial value than the other sex.

20. A method of selecting non-transgenic females of a biological species, the method comprising:
providing a first strain of the biological species genetically engineered to comprise a conditional W-linked genetic lethal circuit;
providing a second strain of the biological species genetically engineered to comprise a conditional Z-linked genetic lethal circuit;
performing a first cross mating males of the first strain and females of the first strain under conditions effective to express the conditional W-linked genetic lethal circuit, thereby producing non-transgenic male progeny; and
mating the non-transgenic progeny of the first cross with females of the second strain under conditions effective to express the conditional Z-linked genetic lethal circuit, thereby producing non-transgenic females.

21. The method of claim 20, wherein the Z-linked genetic lethal circuit is the same as the W-linked genetic lethal circuit.

22. The method of claim 20, wherein the Z-linked genetic lethal circuit is different than the W-linked genetic lethal circuit.

23. The system of claim 20, wherein the biological species is a pest species.

24. The system of claim 20, wherein the biological species is a species in which one sex has greater commercial value than the other sex.

25. A method of selecting non-transgenic females of a biological species, the method comprising:
providing females of the biological species genetically engineered to comprise a conditional Z-linked genetic lethal circuit; and
mating wild-type males with females of the biological species under conditions effective to express the conditional Z-linked genetic lethal circuit, thereby producing non-transgenic females.


SEQUENCE LISTING (8 sequences)

SEQ ID NO: 1 - DNA, 9544 bp, Artificial
  Plasmid
ggcccggtacgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggctagaactagtgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacactagtggatccagcggccgcacctgcaggccggccgttaacacgcgtccgcggtctagactcgaggattccagatctggtaccgggccgctgtatggatatttgcagggccgcagaattctctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggaagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagatccgagctcgtaaactcgactttcacttttctctatcactgatagggagtggtaaactcgactttcacttttctctatcactgatagggagtggtaaactcgactttcacttttctctatcactgatagggagtggtaaactcgaagcgccggatccgtcgagggaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagccgatctaaaaggtaggttcaaccactgatgcctaggcacaccgaaacgactaaccctaattcttatcctttacttcaggcggccgggctcgagggtaccaacttaaaaaaaaaaatcaaaatggtcagccgtttggataaatccaaagttattaattccgctttggaattgttgaatgaagttggtattgaaggtttgacaacacgtaaattggctcaaaaattgggtgttgaacaaccaacattgtattggcatgttaaaaataaacgtgctttgttggatgctttggctattgaaatgttggaccgtcatcatacacatttttgcccattggaaggcgaatcctggcaagatttcttgcgtaataatgccaaatccttccgttgtgctttgttgtcccatcgtgatggtgccaaggttcatttgggcacacgtccaacagaaaaacaatatgaaacattggaaaatcaattggctttcttgtgtcaacaaggcttcagcttggaaaatgctttgtatgctttgagcgccgttggtcattttacattgggctgtgtgttggaagatcaagaacatcaagtcgctaaagaagaacgtgaaacaccaacaacagattcgatgcccccattgttgcgtcaagcaattgaattgttcgatcatcaaggagccgaaccagcattcttgttcggcttggaattgattatttgtggattggaaaaacaattgaaatgtgaatcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagtaagcttggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctcgagcatgcgcaaatttaaagcgctgatatcgatcgcgcgcagatctgtcatgatgatcattgcaattctgcagtcgacggtacccgatcttgtcgccggaacgcagcgacagagattccaatgtgtccgtatctttcaggcttttgcccttcagttccagacgaagcgactggcgattcgcgtgtggggtctgcttcagggtcttgtgaattagggcgcgcagatcgccgatgggcgtggcgccggagggcaccttcaccttgccgtacggcttgctgttcttcgcgttcaaaatctccagctccattttgctttcggtgcgcttgcaatcagtactgtccaaaatcgaaaatcgccgaaccgtagtgtgaccgtgcggggctctgcgaaaataaacttttttaggtatatggccacacacggggaaagcacagtggattatatgttttaatattataatatgcaggttttcattacttatccagatgtaagcccacttaaagcgatttaacaattatttgccgaaagagtaaaaacaaatttcacttaaaaatggattaagaaaagcttgtgtaagattatgcgcagcgttgccagatagctccatttaaaacacttcaaaaacaataagttttgaaaatatatacataaatagcagtcgttgccgcaacgctcaacacatcacacttttaaaacaccctttacctacacagaattactttttaaatttccagtcaagctgcgagtttcaaaattatagccggtagagaagacagtgctatttcaaaagcaaactaaataaacaccaatcctaacaagccttggacttttgtaagtttagatcaaaggtggcattgcattcaatgtcatggtaagaagtaggtcgtctaggtagaaatcctcattcagccggtcaagtcagtacgagaaaggtctcaatttgaaattgtcttaaaaatattttattgttttgtactgtggtgagtttaaacgaaaaacacaaaaaaaaagtgatacacagaaatcataaaaaattttaatacaaggtattcgtacgtatcaaaaacatttcggcacaattttttttctctgtactaaagtgttacgaacactacggtattttttagtgattttcaacggacaccgaaggtatataaacagcgttcgcgaacggtcgccttcaaaaccaattgacatttgcagcagcaagtacaagcagaaagtaaagcgcaatcagcgaaaaatttatacttaattgttggtgattaaagtacaattaaaagaacattctcgaaagtcacaagaaacgtaagtttttaactcgctgttaccaattagtaataagagcaacaagacgttgagtaatttcaagaaaaactgcatttcaaggtctttgttcggccattttttttttattcaacgctctacgtaattacaaaataagaaattggcagccacgcatcttgttttcccaatcaattggcatcaaaacgcaaacaaatctataaataaaacttgcgtgttgattttcgccaagatttattggcaaattgtgaaattcgcagtgacgcatttgaaaattcgagaaatcacgaacgcactcgagcatttgtgtgcatgttattagttagttagttctttgcttaattgaagtattttaccaacgaaatccacttatttttagctgaaatagagtaggttgcttgaaacgaaagccacgtctggaaaatttcttattgcttagtagttgtgacgtcaccatatacacacaaaataatgtgtatgcatgcgtttcagctgtgtatatatacatgcacacactcgcattatgaaaacgatgacgagcaacggaacaggtttctcaactacctttgttcctgtttcttcgctttcctttgttccaatattcgtagagggttaataggggtttctcaacaaagttggcgtcgataaataagtttcccatttttattccccagccaggaagttagtttcaatagttttgtaatttcaacgaaactcatttgatttcgtactaattttccacatctctattttgacccgcagaataatccaaaatgcagatcggggatcccaccccacccaagaagaagcgcaaggtggaggacgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcggtcgactctagaggatccccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcactagagcggccgccaccgcggtggagctccagcttttgttccctttagtgagggttaattagatcggccggccttggcgcgcctagatcttaatacgactcactatagggcgaattgggtaccg

SEQ ID NO: 2 - DNA, 12394 bp, Artificial
  Plasmid
ggcccggtacgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggctagaactagtgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacactagtggatccagcggccgcacctgcaggccggccgttaacacgcgtccgcggtctagactcgaggattccagatctggtaccgggccgctgtatggatatttgcagggccgcagaattctctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggaagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagatccgagctcgtaaactcgactttcacttttctctatcactgatagggagtggtaaactcgactttcacttttctctatcactgatagggagtggtaaactcgactttcacttttctctatcactgatagggagtggtaaactcgaacggatccgtcgagggaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagccatggtaattttctttacgtatatcaagtgttacggctccatttttctttagatatttccagtatagttttttattaccaacatttaaaaacaaattttagaaagcatactgttgggatttaaatgatttttttattaaaaagtgagacaaaattttcaatacagttttaataatggcaaaagaaaatatactgaaaacgttgcattttccaagaggaacaaactacaatcaacatactatgtcttggtttgaagaagaagttgtgacatattgggcagttaaaacaagactataacagtgagtattataaaaaaattgttaaaataacatattcctatatatatttatagcattttaaataaatattaaacatttatttatcattaagttataagacatatattcaaatattgttgtaacagctgtaaaaacaagttagttaattgttattattcaggttctggttaaactccaggtcatgaaattgtgtctcttctataagataagtccctggatcttctggagtaagagtggtaggattggcttatcagttaaacaaatgttctgtatcatgtggtttgaccacatactggacaaatattagggacggcacggtcaatacgagactagtatgcatcgagactattgatccaccccaagcgtagttgttccagagcttcttttgtagacctcggtagattctgttctataaaaaccaagtatcatgcttcttactgatgagaattaacgagaacgtaaagcgcctacacatgtttataatgtattcccgcaagcatatgtatacattgtaatctctgtgtgaccattatatttataatctttgatataaaccttattccagggtactaaagatatattttaattttatttcttttgaatgtctgttgcaaaatatgtaaatatataaaaacttttatgaaaatattatttaagtaaagatataaaatagttgaaaaaatctttgaattgtagaaaaatagtccgctcccctacttgatgcccatattcgcattcggtgtagtcatcttgttccatgctttaagccgcttctcacaagtattccagtaagaggtcacccgtgttaattaggcatagctatcagtttattttagcactatttgatcatctaaatccctgctcctgctaactaccaccttgtacttaaagtacataaaatttggtcttcagcatcgttcttcgaggggcggtcataatattttttatattttaaggagtgagatcgaacgtttttaaagtgctgtaattttgctcgaataggtagtacatctcgttttaaaatctaacacttggaaacctattttgtgcccttcaattaataattatcatgagcataatatctctctactggccttagctccgggctttttggagaaaaaaagtcggcacataatgaagtcttataaatgaaaacagtctttcttgtaaacgttccttgatttatatttatagagcctgttgtaacaaataattagcttcttaaaaagaacttgactgattttgggtcctaaatttttcttcgacattctcttgagcatcagcaacataaaattttttattaggtagttgcaaaaaaactgccatgattagtcatccattatgcaacaagaatactttttcaagaaaacttgattttaatttccgtactctgttttaggcctctaattttttgagcaacattcctgtcaaaaactttttgagccttgtacgtttttaaacctgcatcagctttaacttttcgtaccaaatagtccgaccaatgagctaaccgggcttttctacctaatatgttggcagctcttttgaaaatattttgagacatcatgtgaaccattgcttccacatgaaccagttttctctgggtactgtttgaaaaacataggaaacagcttggcggcaaacctttgaatgcttgggcaacttttgtgggacaaagttttggtttgttgaaaatatttaataatttattaggcacttttttctggtccctcatttaaatcggattaccaatttcatttgtttttaattaaacattataaattatatgttttacatgtttcactaaacgtgtgttactaatttttcgatctcactccttttataccgtattatattaattctatactgtaattaaagttattttcaaattgttgcaatatttatttagcaaaatgttttcataacgtgaaattgtatgttatttttagaaaaaatgtatattaagttctcaaattcatattgtttattcatttagattcccttgaacaaaaggggtttggtattttgtaaaaaattactatattatttaataagtagttaagattaatgaatttcagtgaataataaaagctcaacaatcaacatactaacattttgaagatcagcaatattaatctatcaacactaattataattaacgacaatcaacataccatagaaaaaaggataatggataatgaatacaaaactacaatcaacattttcctcagggcaacacacatctaggttttgcaaggatcaacaaatcaagagtgcattaaaaataacaacaatcaacataccataattgaagatgttgcaaatattgaaaatttttattaaaaacatttaaaatttacttaaatttttcctttaaacgcaaataaaaagaaaacttaaattattctattgcaaacagaaaaaatcccaaattaaaatttatttaaaaattatttttgttataaaacaaatctaaaatctatttaattttaaaaataattaaaaaaaaacataaacgtgttaaaacaatttcacagcttaaaaatatcgataaaaaatatataatttttaataatttattttaattaatcatctttatcaacatacaaaatgatagatagattttaaaaggatcgaggttgcatgtatgataaatttattattcttttctatgtttaggtcagccgtttggataaatccaaagttattaattccgctttggaattgttgaatgaagttggtattgaaggtttgacaacacgtaaattggctcaaaaattgggtgttgaacaaccaacattgtattggcatgttaaaaataaacgtgctttgttggatgctttggctattgaaatgttggaccgtcatcatacacatttttgcccattggaaggcgaatcctggcaagatttcttgcgtaataatgccaaatccttccgttgtgctttgttgtcccatcgtgatggtgccaaggttcatttgggcacacgtccaacagaaaaacaatatgaaacattggaaaatcaattggctttcttgtgtcaacaaggcttcagcttggaaaatgctttgtatgctttgagcgccgttggtcattttacattgggctgtgtgttggaagatcaagaacatcaagtcgctaaagaagaacgtgaaacaccaacaacagattcgatgcccccattgttgcgtcaagcaattgaattgttcgatcatcaaggagccgaaccagcattcttgttcggcttggaattgattatttgtggattggaaaaacaattgaaatgtgaatcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagtaagcttggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctcgagcatgcgcaaatttaaagcgctgatatcgatcgcgcgcagatctgtcatgatgatcattgcaattctgcagtcgacggtacccgatcttgtcgccggaacgcagcgacagagattccaatgtgtccgtatctttcaggcttttgcccttcagttccagacgaagcgactggcgattcgcgtgtggggtctgcttcagggtcttgtgaattagggcgcgcagatcgccgatgggcgtggcgccggagggcaccttcaccttgccgtacggcttgctgttcttcgcgttcaaaatctccagctccattttgctttcggtgcgcttgcaatcagtactgtccaaaatcgaaaatcgccgaaccgtagtgtgaccgtgcggggctctgcgaaaataaacttttttaggtatatggccacacacggggaaagcacagtggattatatgttttaatattataatatgcaggttttcattacttatccagatgtaagcccacttaaagcgatttaacaattatttgccgaaagagtaaaaacaaatttcacttaaaaatggattaagaaaagcttgtgtaagattatgcgcagcgttgccagatagctccatttaaaacacttcaaaaacaataagttttgaaaatatatacataaatagcagtcgttgccgcaacgctcaacacatcacacttttaaaacaccctttacctacacagaattactttttaaatttccagtcaagctgcgagtttcaaaattatagccggtagagaagacagtgctatttcaaaagcaaactaaataaacaccaatcctaacaagccttggacttttgtaagtttagatcaaaggtggcattgcattcaatgtcatggtaagaagtaggtcgtctaggtagaaatcctcattcagccggtcaagtcagtacgagaaaggtctcaatttgaaattgtcttaaaaatattttattgttttgtactgtggtgagtttaaacgaaaaacacaaaaaaaaagtgatacacagaaatcataaaaaattttaatacaaggtattcgtacgtatcaaaaacatttcggcacaattttttttctctgtactaaagtgttacgaacactacggtattttttagtgattttcaacggacaccgaaggtatataaacagcgttcgcgaacggtcgccttcaaaaccaattgacatttgcagcagcaagtacaagcagaaagtaaagcgcaatcagcgaaaaatttatacttaattgttggtgattaaagtacaattaaaagaacattctcgaaagtcacaagaaacgtaagtttttaactcgctgttaccaattagtaataagagcaacaagacgttgagtaatttcaagaaaaactgcatttcaaggtctttgttcggccattttttttttattcaacgctctacgtaattacaaaataagaaattggcagccacgcatcttgttttcccaatcaattggcatcaaaacgcaaacaaatctataaataaaacttgcgtgttgattttcgccaagatttattggcaaattgtgaaattcgcagtgacgcatttgaaaattcgagaaatcacgaacgcactcgagcatttgtgtgcatgttattagttagttagttctttgcttaattgaagtattttaccaacgaaatccacttatttttagctgaaatagagtaggttgcttgaaacgaaagccacgtctggaaaatttcttattgcttagtagttgtgacgtcaccatatacacacaaaataatgtgtatgcatgcgtttcagctgtgtatatatacatgcacacactcgcattatgaaaacgatgacgagcaacggaacaggtttctcaactacctttgttcctgtttcttcgctttcctttgttccaatattcgtagagggttaataggggtttctcaacaaagttggcgtcgataaataagtttcccatttttattccccagccaggaagttagtttcaatagttttgtaatttcaacgaaactcatttgatttcgtactaattttccacatctctattttgacccgcagaataatccaaaatgcagatcggggatcccaccccacccaagaagaagcgcaaggtggaggacgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcggtcgactctagaggatccccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcactagagcggccgccaccgcggtggagctccagcttttgttccctttagtgagggttaattagatcggccggccttggcgcgcctagatcttaatacgactcactatagggcgaattgggtaccg

SEQ ID NO: 3 - DNA, 20 bp, Artificial
  Primer
gccgcagaattctctctatc

SEQ ID NO: 4 - DNA, 21 bp, Artificial
  Primer
cttagctttcgcttagcgacg

SEQ ID NO: 5 - DNA, 20 bp, Artificial
  Primer
tgcaggtgacctgggaatag

SEQ ID NO: 6 - DNA, 20 bp, Artificial
  Primer
gtgagaccacttgaccacag

SEQ ID NO: 7 - DNA, 18 bp, Artificial
  Primer
cgcgacgatagacagcgg

SEQ ID NO: 8 - DNA, 20 bp, Artificial
  Primer
gagagcaatgcgctcgttgc